SEQUENCE LISTING (37 sequences)

SEQ ID NO: 1 - protein, 442 aa, Homo sapiens
MALPVTALLLPLALLLHAARPEQKLISEEDLHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 587 aa, Homo sapiens
MALPVTALLLPLALLLHAARPEQKLISEEDLHMQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 587 aa, Homo sapiens
MALPVTALLLPLALLLHAARPEQKLISEEDLHMQDPYVKEAENLKKYFNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 587 aa, Homo sapiens
MALPVTALLLPLALLLHAARPEQKLISEEDLHMQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRSQMLFRGRRASQHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 587 aa, Homo sapiens
MALPVTALLLPLALLLHAARPEQKLISEEDLHMQDPYVKEAENLKKYFNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRSQMLFRGRRASQHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 437 aa, Homo sapiens
MALPVTALLLPLALLLHAARPEQKLISEEDLHMQDPYVKEAENLKKYFNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRSQMLFRGRRASQHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGR

SEQ ID NO: 7 - protein, 480 aa, Homo sapiens
MALPVTALLLPLALLLHAARPEQKLISEEDLHMQDPYVKEAENLKKYFNAGHSDVQDNGTLFLGILKNWHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 502 aa, Homo sapiens
MALPVTALLLPLALLLHAARPEQKLISEEDLHMDRKIMQSQIVSFYFKLFKNFKDDQSIQKSVETIKEDMNVKFFNSNKKKRDDFEKLTNYHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 468 aa, Homo sapiens
MALPVTALLLPLALLLHAARPEQKLISEEDLHMPAAKTGKRKRSQMLFRGRGRRASQHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 572 aa, Homo sapiens
MALPVTALLLPLALLLHAARPEQKLISEEDLHMNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRSQMLFRGRRASQHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 575 aa, Homo sapiens
MALPVTALLLPLALLLHAARPEQKLISEEDLHMQDPYVKEAENLKKYFNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 578 aa, Homo sapiens
MALPVTALLLPLALLLHAARPEQKLISEEDLHMQDPYVKEAENLKKYFNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRSQMHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 579 aa, Homo sapiens
MALPVTALLLPLALLLHAARPEQKLISEEDLHMQDPYVKEAENLKKYFNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRSQMLHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 580 aa, Homo sapiens
MALPVTALLLPLALLLHAARPEQKLISEEDLHMQDPYVKEAENLKKYFNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRSQMLFHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 581 aa, Homo sapiens
MALPVTALLLPLALLLHAARPEQKLISEEDLHMQDPYVKEAENLKKYFNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRSQMLFRHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 582 aa, Homo sapiens
MALPVTALLLPLALLLHAARPEQKLISEEDLHMQDPYVKEAENLKKYFNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRSQMLFRGHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 586 aa, Homo sapiens
MALPVTALLLPLALLLHAARPEQKLISEEDLHMQDPYVKEAENLKKYFNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRSQMLFRGRRASHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 735 aa, Homo sapiens
MALPVTALLLPLALLLHAARPEQKLISEEDLHMQDPYVKEAENLKKYFNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRSQMLFRGRRASQGGGGSQDPYVKEAENLKKYFNAGHSDVQDNGTLFLGILKNWKEESDRKIMQSQIVSFYFKLFKNFKDDQSIQKSVETIKEDMNVKFFNSNKKKRDDFEKLTNYSVTDLNVQRKAIDELIQVMAELSPAAKTGKRKRSQMLFRGRRASQHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 571 aa, Homo sapiens
MALPVTALLLPLALLLHAARPEQKLISEEDLHMAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 571 aa, Homo sapiens
MALPVTALLLPLALLLHAARPEQKLISEEDLHMAPARSPSPSTQPWEHVNAIQEARRLLNLSRDTAAEMNETVEVISKMFDLQEPTCLQTRLELYKQGLRGSLTKLKGPLTMMASHYKQHCPPTPETSCATQIITFESFKENLKDFLLVIPFDCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 571 aa, Homo sapiens
MALPVTALLLPLALLLHAARPEQKLISEEDLHMAPARSPSPSTQPWEHVNAIQEARRLLNLSRDTAAEMNETVEVISEMFKLQEPTCLQTRLELYKQGLRGSLTKLKGPLTMMASHYKQHCPPTPETSCATQIITFESFKENLKDFLLVIPFDCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 571 aa, Homo sapiens
MALPVTALLLPLALLLHAARPEQKLISEEDLHMAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLAVIPFDCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 571 aa, Homo sapiens
MALPVTALLLPLALLLHAARPEQKLISEEDLHMAPARSPSPSTQPWEHVNAIQEARRLLNLSRDTAAEMNETVEVISEMFKLQEPTCLQTRLELYKQGLRGSLTKLKGPLTMMASHYKQHCPPTPETSCATQIITFESFKENLKDFLLVIPADCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 571 aa, Homo sapiens
MALPVTALLLPLALLLHAARPEQKLISEEDLHMAPARSPSPSTQPWEHVNAIQEARRLLNLSRDTAAEMNETVEVISKMFKLQEPTCLQTRLELYKQGLRGSLTKLKGPLTMMASHYKQHCPPTPETSCATQIITFESFKENLKDFLLVIPFDCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 571 aa, Homo sapiens
MALPVTALLLPLALLLHAARPEQKLISEEDLHMAPARSPSPSTQPWEHVNAIQEARRLLNLSRDTAAEMNETVEVISEMFKLQEPTCLQTRLELYKQGLRGSLTKLKGPLTMMASHYKQHCPPTPETSCATQIITFESFKENLKDFLAVIPFDCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 571 aa, Homo sapiens; identical to SEQ ID NO: 23

SEQ ID NO: 27 - protein, 571 aa, Homo sapiens
MALPVTALLLPLALLLHAARPEQKLISEEDLHMAPARSPSPSTQPWEHVNAIQEARRLLNLSRDTAAEMNETVEVISKMFKLQEPTCLQTRLELYKQGLRGSLTKLKGPLTMMASHYKQHCPPTPETSCATQIITFESFKENLKDFLAVIPFDCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 571 aa, Homo sapiens
MALPVTALLLPLALLLHAARPEQKLISEEDLHMAPARSPSPSTQPWEHVNAIQEARRLLNLSRDTAAEMNETVEVISKMFKLQEPTCLQTRLELYKQGLRGSLTKLKGPLTMMASHYKQHCPPTPETSCATQIITFESFKENLKDFLAVIPADCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 571 aa, Homo sapiens
MALPVTALLLPLALLLHAARPEQKLISEEDLHMAPARSPSPSTQPWEHVNAIQEARRLLNLSRDTAAEMNETVEVISEMFKLQEPTCLQTRLELYKQGLRGSLTKLKGPLTMMASHYKQHCPPTPETSCATQIITFESFKENLKDFLAVIPADCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 571 aa, Homo sapiens
MALPVTALLLPLALLLHAARPEQKLISEEDLHMAPARSPSPSTQPWEHVNAIQHARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 571 aa, Homo sapiens
MALPVTALLLPLALLLHAARPEQKLISEEDLHMAPARSPSPSTQPWEHVNAIQKARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 571 aa, Homo sapiens
MALPVTALLLPLALLLHAARPEQKLISEEDLHMAPARSPSPSTQPWEHVNAIQRARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 571 aa, Homo sapiens
MALPVTALLLPLALLLHAARPEQKLISEEDLHMAPARSPSPSTQPWEHVNAIQHARRLLNLSRDTAAEMNETVEVISKMFKLQEPTCLQTRLELYKQGLRGSLTKLKGPLTMMASHYKQHCPPTPETSCATQIITFESFKENLKDFLAVIPFDCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 571 aa, Homo sapiens
MALPVTALLLPLALLLHAARPEQKLISEEDLHMAPARSPSPSTQPWEHVNAIQHARRLLNLSRDTAAEMNETVEVISKMFKLQEPTCLQTRLELYKQGLRGSLTKLKGPLTMMASHYKQHCPPTPETSCATQIITFESFKENLKDFLLVIPADCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 571 aa, Homo sapiens
MALPVTALLLPLALLLHAARPEQKLISEEDLHMAPARSPSPSTQPWEHVNAIQHARRLLNLSRDTAAEMNETVEVISEMFKLQEPTCLQTRLELYKQGLRGSLTKLKGPLTMMASHYKQHCPPTPETSCATQIITFESFKENLKDFLAVIPADCWEPVQEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 609 aa, Homo sapiens
MALPVTALLLPLALLLHAARPEQKLISEEDLHMCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 609 aa, Homo sapiens
MALPVTALLLPLALLLHAARPEQKLISEEDLHMCDLPQTHSLGSRRTLMLLAQMRRISLFSCLKDAHDFGFPQEEFGNQFQKAETIPVLHEMIQQIFNLFSTKDSSAAWDETLLDKFYTELYQQLNDLEACVIQGVGVTETPLMKEDSILAVEKYFQRITLYLKEKKYSPCAWEVVRAEIMRSFSLSTNLQESLRSKEHMERKCCVECPPCPAPPVAGPSVFLFPPKPKDTLMISRTPEVTCVVVDVSHEDPEVQFNWYVDGVEVHNAKTKPREEQFQSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2026/0315536 A1
(43) Pub. Date: Oct. 15, 2026

(54) CHIMERIC ANTIGEN RECEPTOR T cell AND USE THEREOF

(71) Applicant: Chang Gung University, Taoyuan City (TW)

(72) Inventors: Cheng-Lung Ku, Taoyuan City (TW); JHAN-JIE PENG, Taoyuan City (TW); Han-Po Shih, Taoyuan City (TW); JING-YA DING, Taoyuan City (TW)

(21) Appl. No.: 19/642,154

(22) Filed: Apr. 8, 2026

Related U.S. Application Data
(60) Provisional application No. 63/786,240, filed on Apr. 9, 2025.

(51) Int. Cl. A61K 35/17; A61K 40/11; A61K 40/31; A61P 37/06; C07K 14/705; C07K 16/24; C12N 5/0783

(52) U.S. Cl. CPC A61K 35/17; A61K 40/11; A61K 40/31; A61P 37/06; C07K 14/70503; C07K 16/24; C12N 5/0636; C07K 2317/76

(57) ABSTRACT

A chimeric antigen receptor T cell is provided. The chimeric antigen receptor T cell includes: i) a CAAR backbone as shown in SEQ ID no: 1; and ii) an antigen recognition domain that includes a modified autoantigen or cytokine; the autoantigen or cytokine includes an amino acid sequence as shown in SEQ ID NO: 2 to SEQ ID NO: 37.

### CROSS-REFERENCE TO RELATED PATENT APPLICATION

This application claims the benefit of priority to the U.S. Provisional Patent Application Ser. No. 63/786,240, filed on Apr. 9, 2025, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### STATEMENT REGARDING SEQUENCE LISTING

This application contains a sequence listing in XML format, which has been submitted electronically and is hereby incorporated by reference in its entirety. The sequence listing, created on Mar. 31, 2026, is named 113P000849US-SEQUENCELISTING.xml and is 53,545 bytes in size.

### FIELD OF THE DISCLOSURE

The present invention relates to a chimeric antigen receptor T cell and use thereof, and specifically, to a chimeric antigen receptor T cell for the treatment of autoimmune diseases and use thereof.

### BACKGROUND OF THE DISCLOSURE

Neutralizing anti-interferon-y autoantibodies (nAIGAs) cause an immune disorder associated with severe infections in adults. Gamma interferon is a cytokine produced by T cell and natural killer cells, possessing a strong immune-activating ability. Once gamma interferon loses its function, it can lead to a significant decrease in the host's resistance to mycobacteria and other intracellular pathogens, and may even result in death. However, neutralizing anti-gamma interferon autoantibodies (nAIGA) impairs the gamma interferon (IFN-γ)-mediated immune response, making patients more susceptible to infections with nontuberculous mycobacteria, Penicillium marneffei, and other intracellular pathogens. Current treatments rely on continuous antibiotic therapy, but no therapy currently offers lasting efficacy.

Chimeric autoantibody receptor (CAAR) T cells are an advanced CAR design for targeting autoreactive B cells without compromising overall humoral immunity. CAAR T cells were first developed for the treatment of pemphigus vulgaris (PV), an autoimmune blistering disease caused by autoantibodies targeting the keratinocyte adhesion protein desmoglein 3 (Dsg3). In this design, the CAAR employs Dsg3 autoantigen as the bait, allowing it to recognize the anti-Dsg3 autoantibody B cell receptor (BCR) expressed on the surface of autoreactive B cells. Notably, Dsg3 CAAR T cells demonstrate remarkable efficacy and safety in eliminating engineered target T cells transplanted into mice and effectively ameliorate blistering in a PV-active immune model. Similar results were displayed by muscle-specific tyrosine kinase (Musk)- and anti-N-methyl-D-aspartate (NMDA) receptor (NMDAR)-CAAR T cells, which were developed against autoreactive B cell responsible for autoantibody production in myasthenia gravis and NMDAR encephalitis, respectively.

However, it remains challenging to deploy this strategy for treating diseases induced by autoantibodies against cytokines, such as patients expressing nAIGAs, due to the complications of designing baits mimicking cytokines without inducing biological outcomes controlled by those cytokines.

Therefore, how to improve the therapeutic effect of chimeric antigen receptor T cell against diseases caused by autoantibodies against cytokines through structural design improvements, and thus overcome the aforementioned shortcomings, has become one of the important issues that this research aims to address.

### SUMMARY OF THE DISCLOSURE

The technical problem to be resolved in the present invention is to provide a chimeric antigen receptor T cell and its uses, addressing the shortcomings of the prior art.

To solve the above-mentioned technical problems, one of the technical solutions adopted by the present invention is to provide a chimeric antigen receptor T cell, which includes i) a CAAR backbone as shown in SEQ ID no: 1; and ii) an antigen recognition domain includes a modified autoantigen or cytokine; wherein the autoantigen or cytokine includes one or more amino acid substitutions, deletions, and/or insertions that completely abolish the biological activity of the corresponding wild-type autoantigen or cytokine, while minimizing any reduction in binding to the autoantibody. Specifically, the autoantigen or cytokine includes an amino acid sequence as shown in SEQ ID NO: 2 to SEQ ID NO: 37.

In one embodiment, the cytokine is interferon-γ (IFN-γ), interferon-α (IFN-α) or granulocyte-macrophage colony-stimulating factor (GM-CSF).

In one embodiment, the chimeric antigen receptor T cell comprise a chimeric autoantibody receptor (CAAR) with double mutations of A23Q and H111D.

In one embodiment, the chimeric antigen receptor T cell maintained surface expression in IFN-γ CAAR containing 1-137 amino acids, and exhibited the strongest cytotoxicity in IFN-γ CAAR with a C-terminal extension of 1-131 to 1-142 amino acids.

In one embodiment, the chimeric antigen receptor T cell eliminates patient-derived autoreactive B cells against IFN-γ.

In one embodiment, among variants of the granulocyte-macrophage colony-stimulating factor, a full-length monomeric GM-CSF with mutations in E21H, D45K, L115A, and F119A completely eliminated the interaction with GMR and the induction of GM-CSF signaling.

In one embodiment, among variants of the granulocyte-macrophage colony-stimulating factor, a full-length monomeric GM-CSF with E21H, D48K, L115A, and F119A mutations retains reactivity to patient-derived GM-CSF autoantibodies.

In one embodiment, in the interferon-α, a full-length monomeric IFN-α with R33A and R120E mutations completely eliminates the interaction with IFNAR.

In one embodiment, in the interferon-α, a full-length monomeric IFN-α with R33A and R120E mutations can still react with patient-derived anti-IFN-α autoantibodies.

To solve the above-mentioned technical problems, another one of the technical solutions adopted by the present invention is to provide a Use of a chimeric antigen receptor T cell as described above, for the preparation of a pharmaceutical composition for treating autoantibody diseases.

The present application utilizes an IFN-γ variant that is unresponsive to the IFN-γ receptor (IFN-γR) as bait to construct a chimeric autoantibody receptor (CAAR) T cell that targets autoreactive B cells expressing the nAIGA B cell receptor. Using K562 and Nalm-6 B-cell leukemia models, the results showed that IFN-γ CAAR T cells lacked off-target toxicity, including IFN-γR cross-reactivity toxicity and Fc-mediated toxicity. It is noted that IFN-γ CAAR human T cell can maintain their function in the presence of high concentrations of nAIGA in vitro. In addition, IFN-γ CAAR T cells effectively eliminated autoreactive B cells from cultured peripheral blood mononuclear cells (PBMCs) expressing nAIGA in vitro. In summary, these results suggest that IFN-γ CAAR T cells hold promise as an effective strategy for alleviating nAIGA-related infections by eliminating autoreactive B cells.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

FIGS. 1A to 1F are schematic diagrams showing chimeric autoantibody receptors carrying truncated IFN-γ, retaining surface expression, and exhibiting broad reactivity to nAIGA BCR.

FIG. 1A is a diagram of nAIGA epitopes on monomer IFN-γ. Three commercial anti-IFN-γ mAbs (site II: NI-0501 and EI-001; site III: AMG811) and five nAIGA mAbs (site I: E1; site II: 2B6 and 2A102; site III: 1E8 and 2A101) cloned from patients with nAIGAs were listed.

FIG. 1B is a schematic of IFN-γ CAAR against nAIGA BCR. The composition of IFN-γ variants in CAAR is shown, with a dashed line indicating the AIGA binding region.

FIG. 1C is a representative flow cytometry plot of surface Myc-tagged IFN-γ CAAR expression on transduced human T cells marked by EGFP.

FIGS. 1D to 1F show the expression of IFN-γ CAAR T cell candidates as detected by flow cytometry-based cytotoxicity assays (n=3-5 donors), where FIG. 1D represents E1, FIG. 1E represents 2B6, and FIG. 1F represents 1E8 cells. AF: truncated IFN-γ with A-to-F helix; 1-137: C-terminal extended IFN-γ containing 137 amino acids. Data shown as mean±s.d. One-way ANOVA analysis with Tukey's or Dunnett's multiple comparison tests was performed (the comparisons are shown in FIGS. 1D to 1F, unless otherwise stated. Other processing groups of the CAAR skeleton are compared).

FIGS. 2A to 2C are schematic diagrams illustrating the cytotoxicity of IFN-γ CAAR T cell against target T cell expressing nAIGA BCR.

FIG. 2A is a diagram of cytotoxicity of IFN-γ CAAR T cell against K562 target T cell expressing luciferase, CD19, and the indicated BCRs, with an elevated E:T ratio, which was assessed by luciferase-based cytotoxicity assay (n=4 donors).

FIG. 2B is a diagram of IL-2 and TNF-α secretion from IFN-γ CAAR T cell in response to K562 target T cell (IL-2, n=7 donors; TNF-α, n=4 donors).

FIG. 2C is a diagram of the percentage of proliferating IFN-γ CAAR T cells after co-culturing with K562 target T cells for 72 hours (n=4 donors). Data shown as mean±s.d. One-way ANOVA analysis with Tukey's multiple comparison tests was performed (FIGS. 2A to 2C). Statistical comparison of Backbone vs. AF IFN-γ and vs. 1-137 IFN-γ against K562 E1, K562 2B6, K562 1E8, and K562 control mAb showed significance levels of (ns, p<0.05, p<0.05, ns) and (p<0.05, p<0.05, p<0.05, ns), respectively, across all E:T ratios.

FIGS. 3A to 3J are schematic diagrams illustrating that soluble nAIGAs reduce the cytotoxicity but not the specificity of IFN-γ CAAR T cells.

FIG. 3A is a diagram of upregulation of CD69 in IFN-γ CAAR T cells in response to soluble control mAb, mixed E1, 2B6, and 1E8 nAIGAs (mixed nAIGAs), or K562 target cell expressing 2B6 nAIGA BCR and CD19 (K562 2B6) (n=3 donors).

FIG. 3B is a diagram of the selective killing assay used to evaluate the interference of soluble nAIGAs in the specificity of IFN-γ CAAR T cells.

FIGS. 3C and 3D are diagrams of the selective cytotoxicity of IFN-γ CAAR T cells against (FIG. 3C) K562 wild-type and (FIG. 3D) K562 2B6 in the presence of plasma derived from patients with nAIGAs (n=3 donors). The patients' plasma containing high (>1000 Units), middle (>100 to 1000 Units), or low (>10 to 100 Units) titer of nAIGAs were marked in red, blue, and black, respectively.

FIGS. 3E and 3F are diagrams of the selective cytotoxicity of IFN-γ CAAR T cells against (FIG. 3E) K562 wild-type and (FIG. 3F) K562 2B6 in the presence of mixed nAIGAs (n=6 donors). Data shown as mean±s.d. One-way ANOVA analysis with Tukey's multiple comparison tests was performed (FIGS. 3A, 3C to 3F).

FIGS. 3G to 3J are schematic diagrams illustrating the different AIGA titer ranges in the serum of patients, used to explore the cytolytic ability of IFN-γ CAAR T cells under extremely high AIGA concentration conditions.

FIGS. 4A to 4F are diagrams of IFN-γ CAAR T cells that lack cross-reactivity against IFN-γR.

FIGS. 4A and 4B are diagrams of the reactivity of IFN-γ CAAR T cells against wild-type, IFN-γ R-, and 2B6 nAIGA BCR plus CD19-overexpressed K562 cells (n=3 donors).

FIG. 4A is a diagram of the induction of CD69 expression in IFN-γ CAAR T cells. FIG. 4B is cytotoxicity of IFN-γ CAAR T cells against K562 target cells. FIGS. 4C to 4F are Reactivity of IFN-γ CAAR T cells against autologous PBMCs (n=3 donors). FIG. 4A is a diagram of fold induction of CD69 expression on resting IFN-γ CAAR T cells upon stimulation with autologous PBMCs. Relative number of (FIG. 4B) B cells, (FIG. 4C) NK cells, and (FIG. 4D) monocytes in PBMCs after IFN-γ CAAR T cells exposure. Scale bar: 100 μm. Data shown as mean±s.d. One-way ANOVA analysis with Tukey's multiple comparison tests and Two-way ANOVA analysis with Dunnett's multiple comparison tests were performed in (FIG. 4A, FIG. 4B, and FIG. 4C) and (FIG. 4D to FIG. 4F), respectively.

FIGS. 4G to 4I are diagrams of cells expressing the FcγIa receptor, which are able to capture circulating immunoglobulin G (IgG) with high affinity.

FIGS. 5A and 5B are diagrams of IFN-γ CAAR T cells that can eliminate autoreactive memory B cells against IFN-γ from patients with nAIGA disease.

FIG. 5A is a diagram of nAIGA production by ex vivo differentiated B cells from the nAIGA patient's PBMCs. Replicate cultures of total PBMCs were seeded in 96-well plates with 10⁵ cells per well density and co-cultured with autologous T cells expressing CAAR backbone or IFN-γ CAAR at a 1:30 E:T ratio in memory B cell differentiation medium for 10 days. Each dot represents the result from one well. FIG. 5B is a diagram of the number of clonable nAIGA memory B cells per 10⁶ PBMCs in patients with nAIGAs. N/A: not applicable. Data shown as mean±s.d. Kruskal-Wallis test with Dunn's multiple comparisons test was performed (FIG. 5A).

FIGS. 6A to 6H are diagrams of specific target B cells for the production of anti-GM-CSF autoantibodies by GM-CSF CAAR.

FIGS. 7A to 7G are diagrams of B cells that produce anti-type I interferon autoantibodies from IFN-α CAAR.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

Unless otherwise stated, the material(s) used in any described embodiment is/are commercially available material(s) or may be prepared by methods known in the related art, and the process(es) or operation(s) involved in any described embodiment is/are conventional process(es) or operation(s) generally known in the related art.

The term “protein” as used herein refers to a biomacromolecule formed by one or multiple chains of amino acid residues.

The term “antibody” and “immunoglobulin” have the same meaning, and are used as equivalents in the present disclosure. The term “antibody” as used herein refers to immunoglobulin molecules and immunologically active portions of the immunoglobulin molecules (i.e., molecules that contain an antigen-binding site for specifically binding an antigen). As such, the term “antibody” not only covers a complete antibody molecule but also includes an antibody fragment or derivative.

An antibody includes, but is not limited to, a monoclonal antibody, a polyclonal antibody, a monospecific antibody, a multi specific antibody (e.g., a bispecific antibody), a monovalent antibody, a multivalent antibody, a complete antibody, a fragment of the complete antibody, a naked antibody, a conjugated antibody, a chimeric antibody, a humanized antibody, or a fully human antibody.

The term “specific binding” as used herein refers to an antigen-binding molecule (e.g., the antibody) typically using high affinity to specifically bind the antigen and a substantially identical antigen, and not using high affinity to bind unrelated antigens. Affinity is typically reflected by an equilibrium dissociation constant (KD), where a lower KD indicates higher affinity. Taking the antibody as an example, the high affinity typically indicates having a KD of about 1×10-7 M or less, about 1×10-8 M or less, about 1×10-9 M or less, about 1×10-10 M or less, 1×10-11 M or less, or 1×10-12 M or less. The equilibrium dissociation constant is calculated as follows: KD=Kd/Ka, where Kd represents a dissociation rate and Ka represents a binding rate. The equilibrium dissociation constant can be measured by using well-known methods in the related art, such as surface plasmon resonance (e.g., Biacore) or equilibrium dialysis.

The term “treatment” as used herein refers to a surgical or therapeutic treatment for the purpose of preventing or slowing (reducing) an undesired physiological or pathological change (e.g., progression of a cancer) in a treated subject. Beneficial or desired clinical results include, but are not limited to, alleviation of symptoms, a decrease in severity of illness, stabilization (i.e., not worsening) of a state of illness, delay or slowing of illness progression, amelioration or palliation of the state of illness, and remission of the state of illness (whether partial or complete and whether detectable or undetectable). Subjects in need of treatment include those already with a disorder or illness, as well as those who are susceptible to a disorder or illness or those who intend to prevent a disorder or illness. When referring to terms such as slowing, alleviation, decrease, palliation, and remission, their meanings also include elimination, disappearance, nonoccurrence, etc.

The term “subject” as used herein refers to an organism that receives treatment for a particular illness or disorder mentioned in the present disclosure. Examples of subjects and patients include mammals, such as human, primates (e.g., monkeys), or non-primates, that receive treatment for an illness or disorder.

The term “effective amount” as used herein refers to an amount of a therapeutic agent that is effective in preventing or alleviating symptoms of an illness or progression of the illness when administered to a cell, tissue, or subject alone or in combination with another therapeutic agent. The effective amount also refers to an amount of a compound that is sufficient to alleviate symptoms (e.g., to treat, cure, prevent, or alleviate related medical disorders), or to increase rates at which such disorders are treated, cured, prevented, or alleviated. When an active ingredient is administered alone to an individual, a therapeutically effective dose refers to an amount of the ingredient alone. When a combination is used, the therapeutically effective dose refers to a combined amount of multiple active ingredients that produce a therapeutic effect (whether administered in combination, sequentially, or simultaneously).

In the following descriptions, advantages and features of the present disclosure will be discussed in detail in conjunction with specific examples. Under the circumstance where specific conditions are not indicated, the examples are carried out according to conventional conditions or according to conditions recommended by a manufacturer. It should be noted that reagents or instruments used in the examples of the present disclosure can be replaced by commercially available products of the same nature. Unless indicated otherwise, cells, vectors, and proteins used for verifying efficacy and activity in the examples of the present disclosure can all be replaced by commercially available products of the same nature or produced by existing techniques.

Embodiment 1: Development of a CAAR bearing a truncated IFN-γ mutant to target autoreactive B cells against IFN-γ.

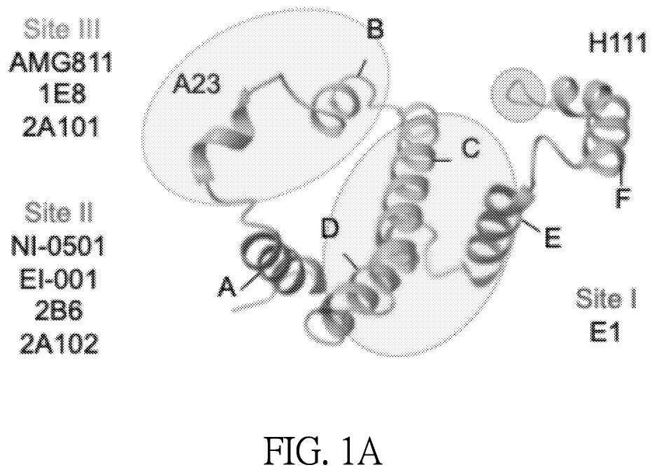

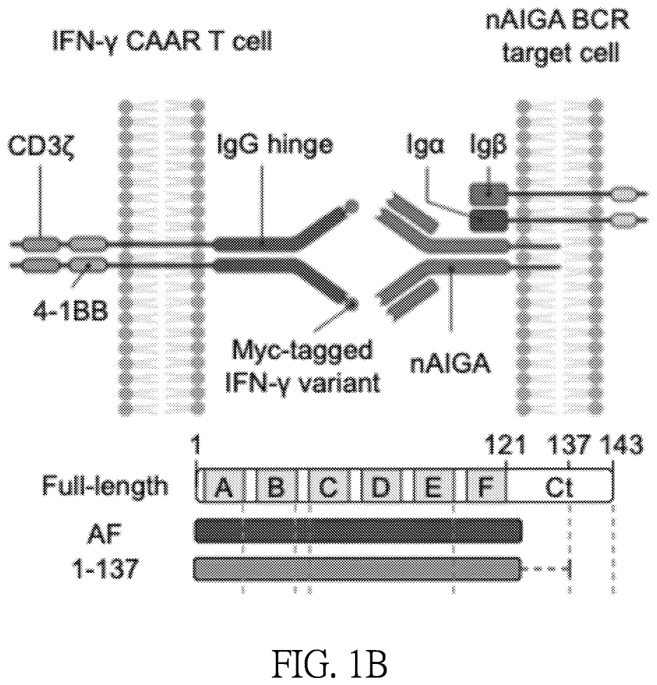

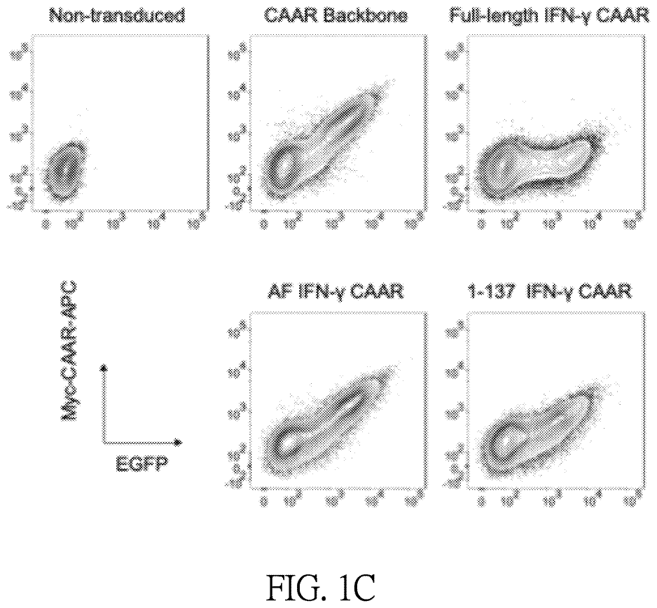

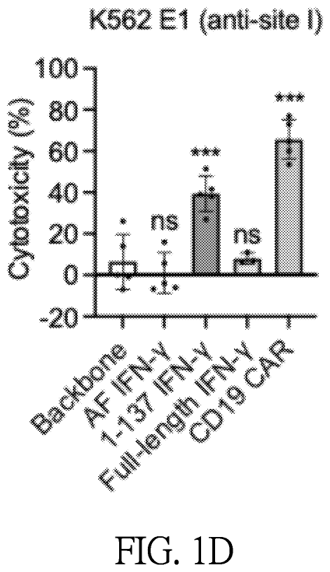

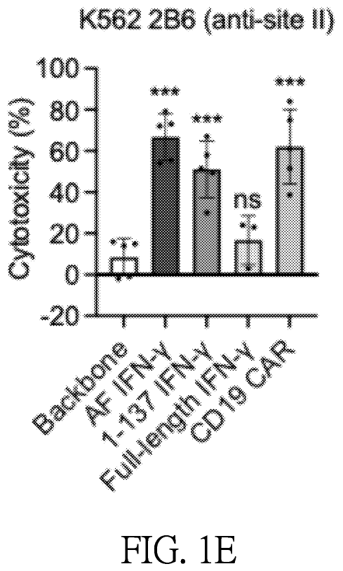

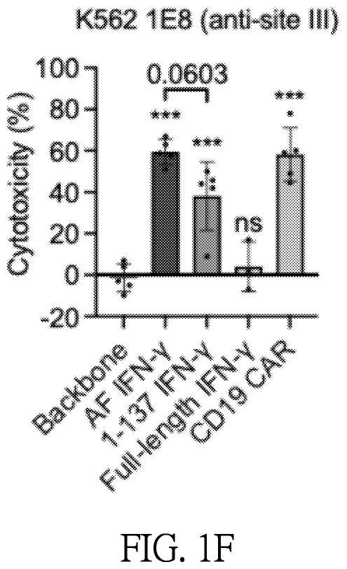

As shown in FIG. 1A, nAIGA BCRs expressed on autoreactive B cells can be categorized into three groups based on the non-overlapping binding epitope on IFN-γ. The epitopes within the flexible C-terminus, C to E helix, and loop between A and B helix (ABL) to C helix are defined as the site I to III epitopes, respectively. As shown in FIG. 1B, the present application therefore postulated that CAAR T cells expressing distinct IFN-γ epitopes to comprehensively cover the nAIGA BCR expressed on autoreactive B cells can be applied to eliminate those autoreactive B cells. To evaluate the reactivity of IFN-γ CAAR, the present application adopted three commercial anti-IFN-γ mAbs (site II: NI-050 and EI-001; site III: AMG811) and five nAIGA mAbs (site I: E1; site II: 2B6 and 2A102; site III: 1E8 and 2A101) cloned from patients with nAIGAs. the present application then optimized the IFN-γ CAAR construct through a stepwise screening process, ensuring receptor signaling was abrogated, confirming nAIGA recognition, and enhancing surface expression for optimal functionality. Since alanine 23 residue in the AB Loop and histidine 111 residue in the F helix of IFN-γ are essential for IFN-γR binding and signaling transduction, the present application substituted both residues with glutamine (Q) and aspartate (D), respectively, to prevent IFN-γR cross-reactivity. The present application confirmed that the two substitutions on IFN-γ completely abrogated IFN-γ signaling in HeLa GAS-luciferase reporter cells.

Of note, IFN-γ can induce functional signaling in a dimer structure. Thus, the present application generated IFN-γ CAARs with either a monomer IFN-γ with point mutations (A23QH111D IFN-γ) or a single chain IFN-γ (scIFN-γ) linked with IFN-γ by the poly-glycine-serine (G₄S) linker (A23QH111D scIFN-γ). By exposing Jurkat cells expressing the indicated CAAR and an NFAT-driven luciferase reporter with coated recombinant IFN-γR1 or THP-1 cells (which highly expressed IFN-γR), the present application confirmed that both IFN-γ CAAR versions failed to induce activation of nuclear factor of activated T cells (NFAT). These results indicate that the present application CAAR designs do not react with IFN-γR.

We next evaluated the activation capacity of IFN-γ CAARs by stimulating Jurkat cells expressing the indicated CAAR and an NFAT-driven luciferase reporter with the coated site I AIGA monoclonal antibody (mAb; clone E1), site II αIFN-γ mAb (clone NI-0501), site III αIFN-γ mAb (clone AMG-811) or a mixed pool of the three αIFN-γ mAbs. Both versions of IFN-γ CAAR responded to all groups of αIFN-γ mAbs. Soluble anti-IFN-γ monoclonal antibodies derived from nAIGA patients did not activate NFAT in Jurkat cells expressing the indicated CAAR and an NFAT-driven luciferase reporter, suggesting the soluble αIFN-γ mAbs are unable to efficiently induce the clustering of IFN-γ CAAR, a critical and initial step for CAR activation. Moreover, the monomer IFN-γ CAAR was chosen for further characterization since it showed a similar and even a trend of higher activation potential compared with the dimer form of CAAR (A23QH111D scIFN-Y).

As shown in FIG. 1C, the present application next introduced the IFN-γ CAAR to primary human T cells by lentivirus and examined its surface expression. The CAAR backbone without IFN-γ could be well presented on the cell surface in human T cells based on staining of the Myc-tag in the N-terminal of CAAR. To the present application's surprise, the present application barely detected surface IFN-γ CAAR in human T cells transduced with a full-length IFN-γ CAAR (FL). Notably, lack of surface expression of IFN-γ CAAR was not due to self-activation-induced endocytosis since the removal of intracellular signaling domains (ICD) (FL w/o ICD) did not improve surface expression. To improve CAAR surface expression, the present application designed several truncated IFN-γ variants as CAARs based on the epitopes of nAIGAs. CAARs containing the A to B helix (AB) of IFN-γ could be detected on the T cell surface, while CAARs with the C to E helix (CE) or C-terminal (Ct, ¹²²PAAKTGKRKRSQMLFRGRRASQ¹⁴³) were not detected. Moreover, the CAARs with IFN-γ lacking the A helix (ABLCt) or C-terminus (AF) could be presented on the T cell surface.

We further examined how these different CAARs with IFN-γ variants respond to coated antibodies composed of a pool of monoclonal nAIGAs against site I to III by analyzing expression of the T cell activation marker CD69. AF IFN-γ CAAR exhibited the most potent activation potential among all IFN-γ variants and was selected for application. Nevertheless, the AB and CE IFN-γ CAARs failed to react with coated nAIGAs even though they could be presented on the cell surface. In contrast, T cells expressed FL IFN-γ as a CAAR were mildly activated by coated nAIGAs, suggesting FL IFN-γ CAAR can still be expressed but with a very low surface expression. Since the site I nAIGAs bind to the C-terminus of IFN-γ, the autoreactive B cells responsible for the site I nAIGA secretion are not targeted by the AF IFN-γ CAAR. Interestingly, sera from patients with nAIGAs fail to recognize the C-terminal-deleted IFN-γ, but remain active to detect truncated forms of IFN-γ covering amino acids 1-131. Thus, the present application speculated that a C-terminal extended IFN-γ variant can serve as a CAAR to target site I nAIGAs.

To examine this, the present application generated a variety of C-terminal-extended IFN-γ variants to cover amino acids 131 to 143 and investigated their performance as a CAAR by examining their surface expression and interaction with the site I nAIGAs. The present application found that CAARs with IFN-γ variants covering amino acid residues 1 to 138 can be presented on the cell surface based on the staining of Myc-tag and a site in nAIGA (E1 clone). However, similar to FL IFN-γ, the CAAR with IFN-γ variant covering amino acid residues 1 to 142 (1-142) exhibited poor surface expression.

We next evaluated the cytotoxic activity of C-terminal-extended IFN-γ CAARs against K562 cells simultaneously expressing indicated autoantibody BCRs for IFN-γ and CD19 antigen. As shown in FIG. 1D and FIG. 1G, in this setting, the present application applied a CD19 CAR as a positive control to quantify the killing ability of CAAR and CD19 CAR T cells. Consistent with the intensity of E1 binding to IFN-γ variants, CAAR with IFN-γ variants covering amino acid residues 1 to 138 could partially deplete K562 cells expressing nAIGA BCRs.

As shown in FIG. 1H and FIG. 1I, among those CAAR with IFN-γ variants, the 1-137 IFN-γ CAAR displayed the most potent cytotoxicity against K562 cells expressing the E1 nAIGA BCR (site I nAIGA) and showed superior cytotoxicity against K562 cells expressing the 1E8 nAIGA BCR (site II nAIGA) and 2B6 nAIGA BCR (site III nAIGA) among the C-terminal extended CAAR T cells. AF and 1-137 IFN-γ CAAR T cells could also target the K562 cells expressing a low-affinity non-neutralizing AIGA BCR such as 2A102 (site II AIGA) and 2A101 (site III AIGA).

Particularly, as shown in FIG. 1G, 1-137 IFN-γ CAAR was less potent in killing target cells expressing a site III nAIGA BCR compared with AF IFN-γ CAAR T cells. In addition, the present application CAAR designs did not kill K562 cells expressing a control antibody BCR, highlighting the specificity of these CAARs in targeting AIGA BCRs. Together, the present application results demonstrate that the truncated IFN-γ mutant can be used for CAAR T cells against B cells expressing AIGA BCRs, and AF and 1-137 IFN-γ CAARs may display different advantages in eliminating autoreactive B cells recognizing distinct epitopes of IFN-γ.

Embodiment 2: IFN-γ CAAR T cells effectively eliminate AIGA BCR-expressing target cells.

To further investigate the efficacy of IFN-γ CAAR T cells on eliminating target cells expressing AIGA BCRs, the present application cultured AF and 1-137 IFN-γ CAAR T cells with K562 cells expressing different AIGA BCRs and CD19 antigen at an elevated effector to target (E:T) ratios. The present application first examined the surface expression and binding of nAIGAs against different epitopes in human T cells. The 1-137 IFN-γ CAAR had a lower surface expression compared to AF IFN-γ CAAR, but 1-137 IFN-γ CAAR displayed a higher E1 nAIGA binding intensity. In contrast, the AF IFN-γ CAAR displayed a trend of binding 2B6 and 1E8 more efficiently.

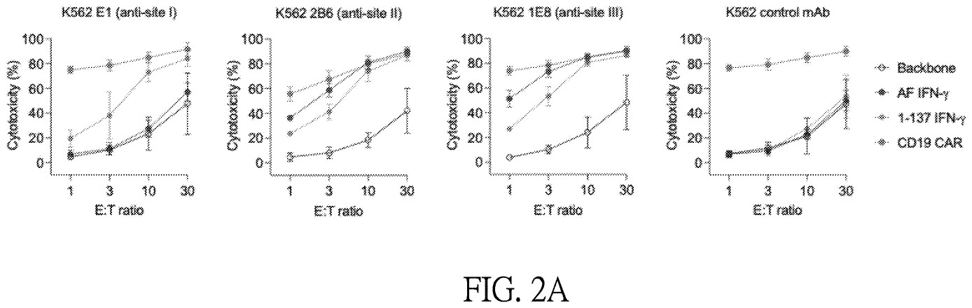

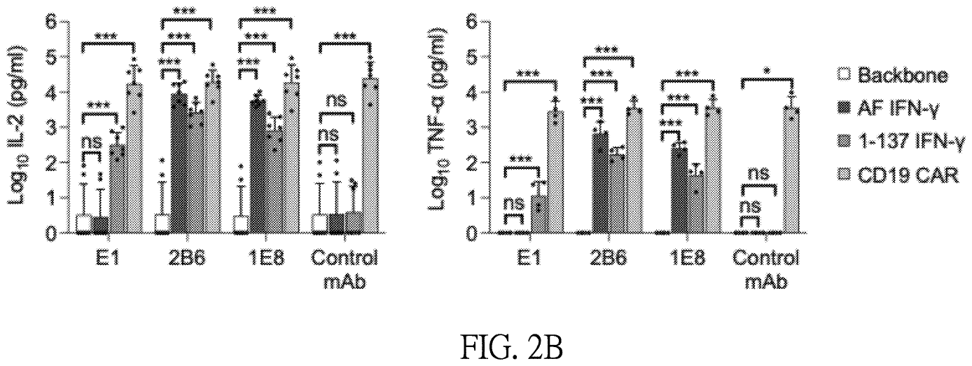

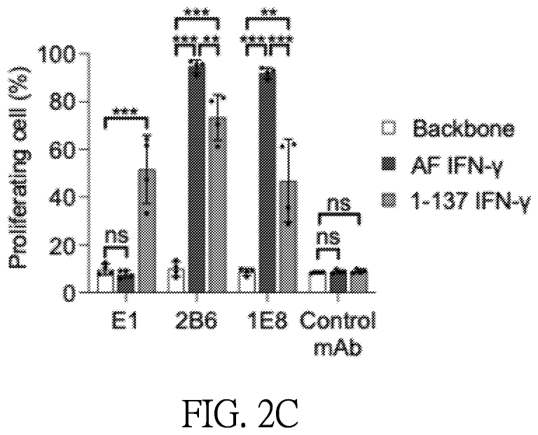

As shown in FIG. 2A, in line with the intensity of nAIGA binding, 1-137 IFN-γ CAAR T cells more effectively eliminated K562 cells expressing the E1 nAIGA BCR in a dose-dependent manner. In contrast, as shown in FIG. 2B, AF IFN-γ CAAR T cells more efficiently eliminated K562 cells expressing the 1E8 or 2B6 nAIGA BCR compared with 1-137 IFN-γ CAAR T cells at low E:T ratios. Both AF and 1-137 IFN-γ CAAR T cells exhibit minimal non-specific cytotoxicity against control target cells expressing control BCR since they displayed similar killing ability compared to T cells expressing CAAR backbone. In addition, AF IFN-γ CAAR T cells secreted interleukin-2 (IL-2) and tumor necrosis factor-α (TNF-α) after engaging with K562 cells expressing the 2B6 or 1E8 nAIGA BCR, but not with K562 cells expressing E1 nAIGA BCR.

In contrast, as shown in FIG. 2C, 1-137 IFN-γ CAAR T cells produced higher levels of IL-2 and TNF-α after engaging with K562 cells expressing E1 nAIGA BCR. In parallel, 1-137 IFN-γ, but not AF, CAAR T cells displayed increased proliferation upon co-culture with K562 cells expressing the E1 nAIGA BCR. Taken together, the present application data indicate that AF IFN-γ CAAR T cells have a higher ability to target cells expressing nAIGA BCRs against the site II and III epitopes of IFN-γ compared to 1-137 IFN-γ CAAR T cells, but 1-137 IFN-γ CAAR T cells are effective for eliminating target cells expressing nAIGA BCRs against site I epitopes.

Embodiment 3: Activity of IFN-γ CAAR T cells in the presence of nAIGAs.

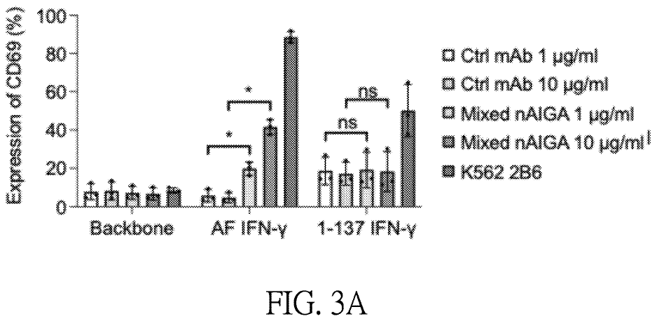

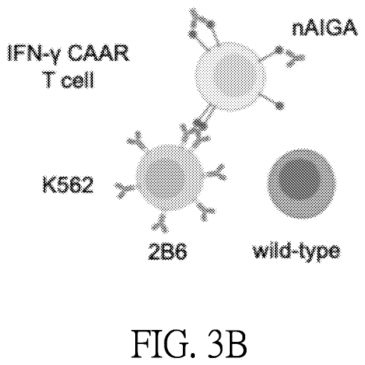

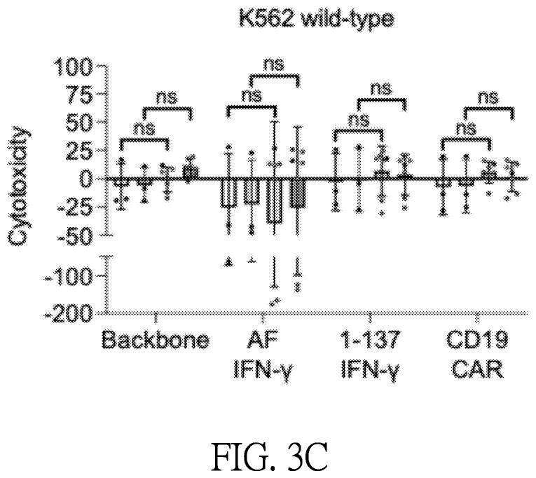

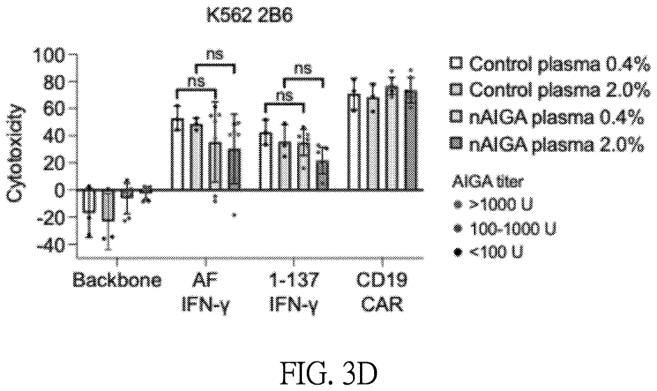

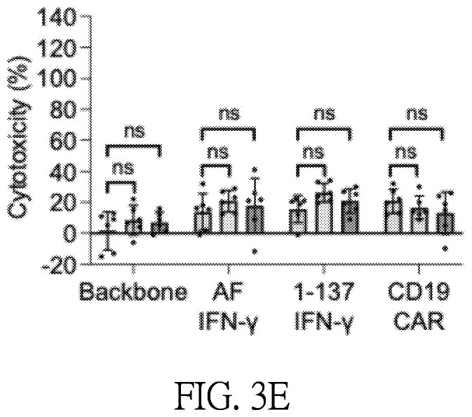

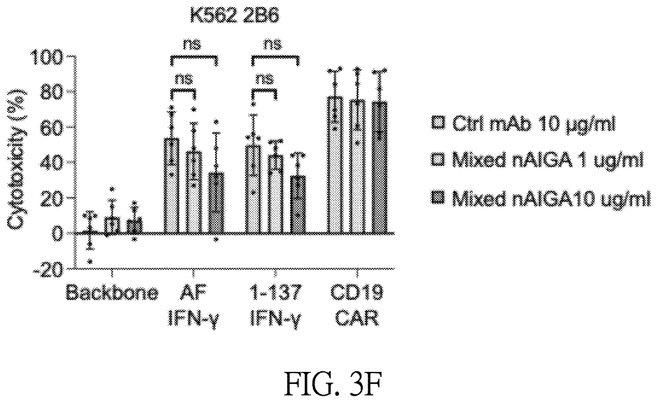

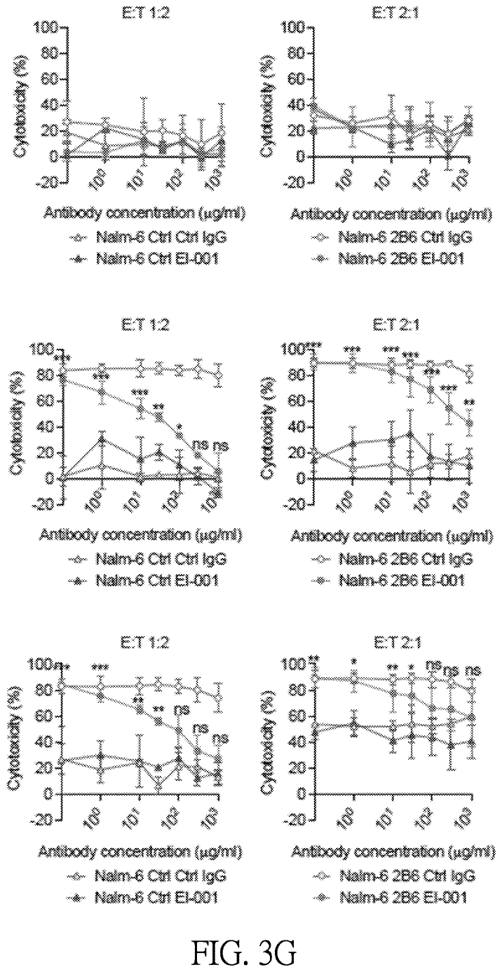

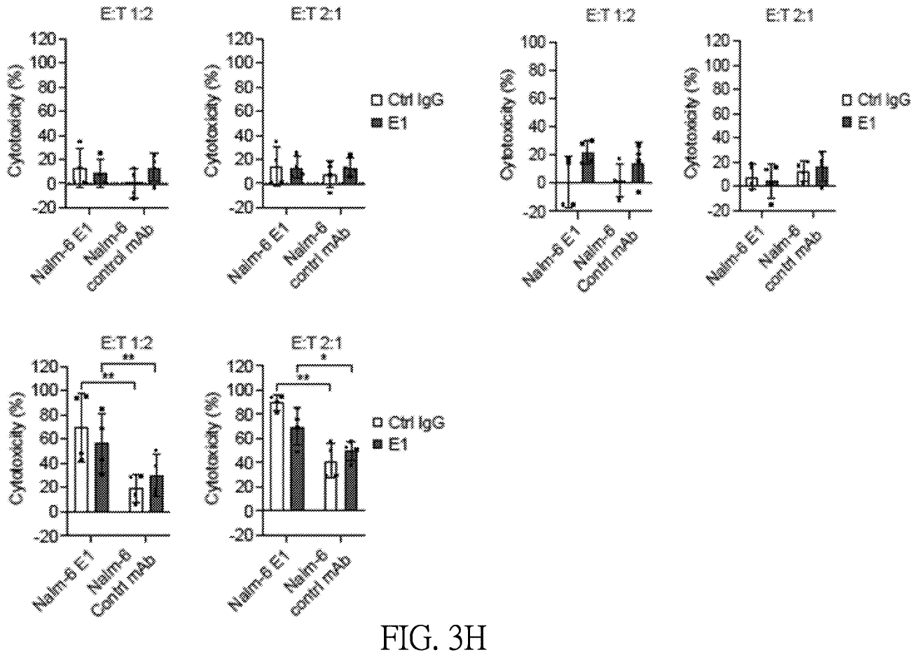

Since soluble nAIGAs in circulation can bind to the IFN-γ CAAR T cells that might elicit unanticipated responses and block the interaction of IFN-γ CAAR with nAIGA BCRs on autoreactive B cells, the present application then analyzed the response of IFN-γ CAAR T cells to soluble nAIGAs. As shown in FIG. 3A, exposure to mixed nAIGAs, a combination of nAIGA mAbs against all sites of IFN-γ (clone E1, 2B6, and 1E8), led to upregulation of CD69 in AF IFN-γ CAAR T cells, but not in 1-137 IFN-γ CAAR T cells. Of note, the induction of CD69 in AF IFN-γ CAAR T cells upon nAIGAs exposure was less compared to the levels induced by K562 target cells expressing the 2B6 nAIGA BCR, suggesting that soluble nAIGAs may not be able to fully activate AF IFN-γ CAAR T cells.

As shown in FIG. 3B, the present application next evaluated the impact of soluble AIGAs on the specificity and potency of IFN-γ CAAR T cells on eliminating their target cells by an in vitro selective killing assay. As shown in FIGS. 3C and 3D, to better recapitulate physiological conditions, the present application mixed wild-type K562 cells with K562 cells expressing the 2B6 nAIGA BCR and then co-cultured them with IFN-γ CAAR T cells or CD19 CAR T cells in the presence of low levels of plasma collected from control human subjects or patients with nAIGAs. Both IFN-γ CAAR T cells and CD19 CAR T cells effectively kill K562 cells expressing the 2B6 nAIGA BCR, but not wild-type K562 cells. Meanwhile, the present application observed a trend, albeit not significant, that an increasing amount of plasma from patients with nAIGAs decreased the cytotoxicity of AF IFN-γ CAAR T cells and 1-137 IFN-γ CAAR T cells.

Referring to FIG. 3E and FIG. 3F, the present application further investigated whether the mixed soluble nAIGAs could impede cytotoxicity of AF IFN-γ CAAR T cells and 1-137 IFN-γ CAAR T cells. Similar to the present application results using plasma from patients, there was a not significant trend that increasing amount of nAIGAs decreased the cytotoxicity of AF IFN-γ CAAR T cells and 1-137 IFN-γ CAAR T cells.

As shown in FIGS. 3G to 3J, The AIGA titer in patients' serum ranged from approximately 2 to 1800 U (1 U corresponding to 1 μg/mL of 2F2 standard AIGA mAb), with a median titer around 120 U. Given this range, the present application explored the cytolytic capacity of IFN-γ CAAR T cells under extremely high AIGA concentration conditions using a concentrated αIFN-γ mAb (clone EI-001) currently in clinical development. Notably, the αIFN-γ mAb (clone EI-001) did not induce toxicity by CAAR backbone-expressing T cells toward either Nalm6 cells expressing the 2B6 nAIGA BCR or Nalm6 cells expressing the control mAb BCR. AF IFN-γ CAAR T cells could reduce Nalm-6 cells expressing the 2B6 nAIGA BCR at concentrations of 10 to 100 μg/ml αIFN-γ mAb (clone EI-001) at a low E:T ratio, while maintaining function at up to 1 mg/ml αIFN-γ mAb at a higher E:T ratio.

However, 1-137 IFN-γ CAAR T cells were less robust than the AF version. Similar results were observed when using neutralizing and non-neutralizing monoclonal AIGAs from site I to site III. Notably, non-neutralizing AIGAs (clones 2A102 and 2A101) had less impact on IFN-γ CAAR T cells, suggesting both AIGA titer and the AIGA composition in patient serum can affect the efficacy of IFN-γ CAAR T cells.

Embodiment 4: Absence of off-target toxicity in IFN-γ CAAR T cells.

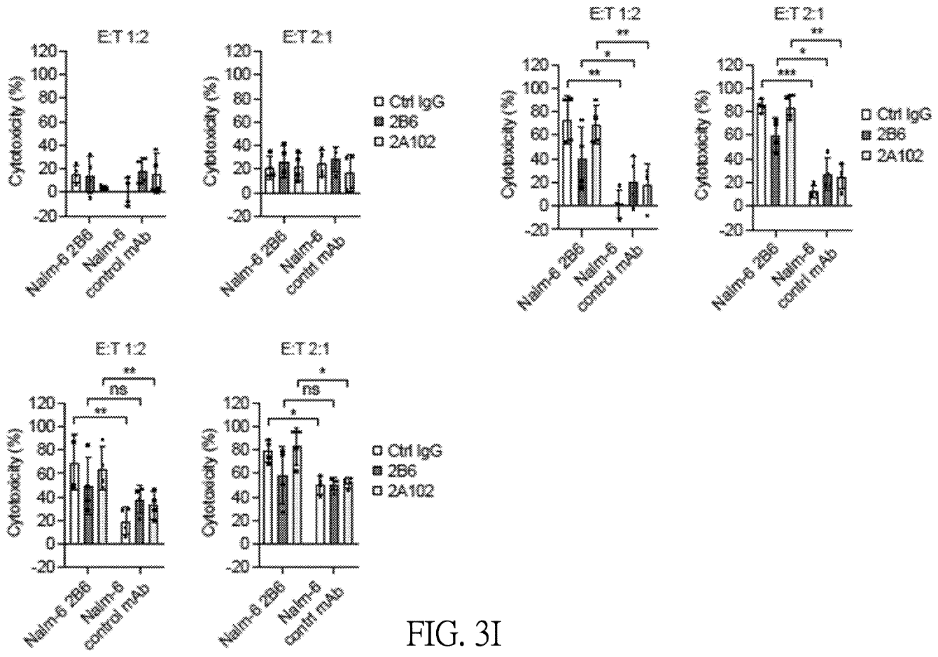

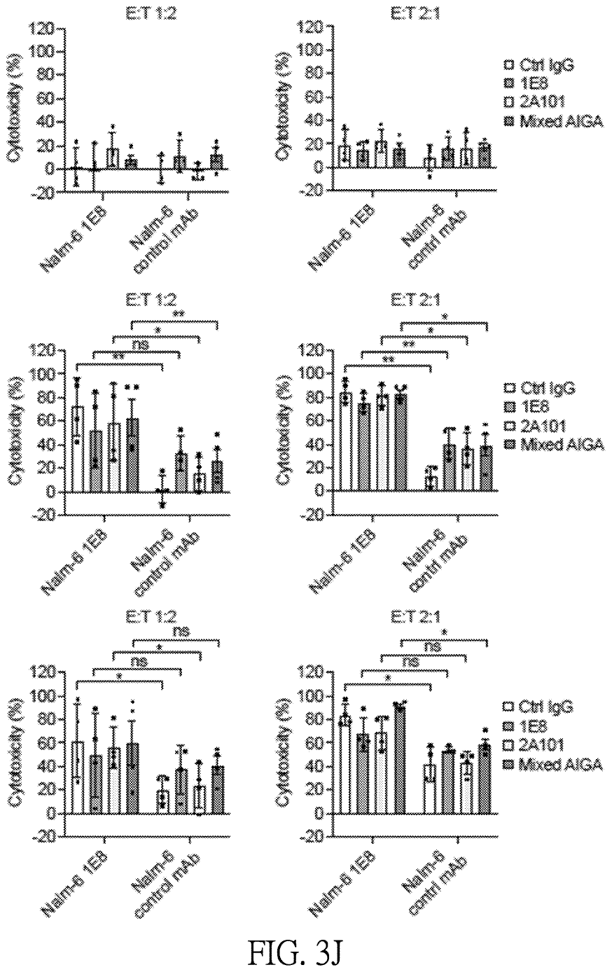

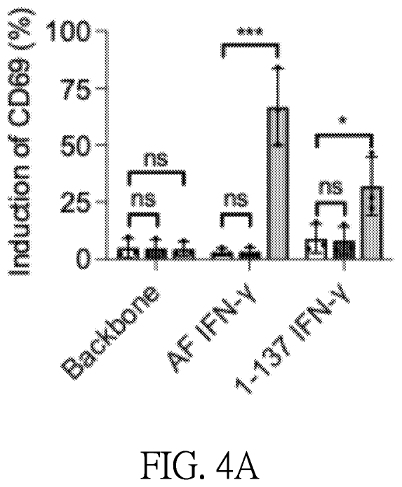

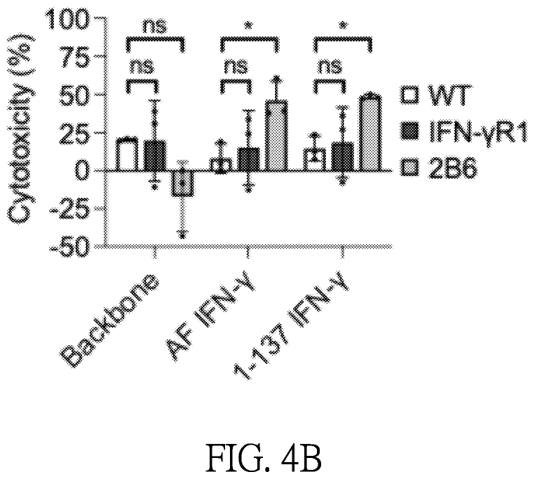

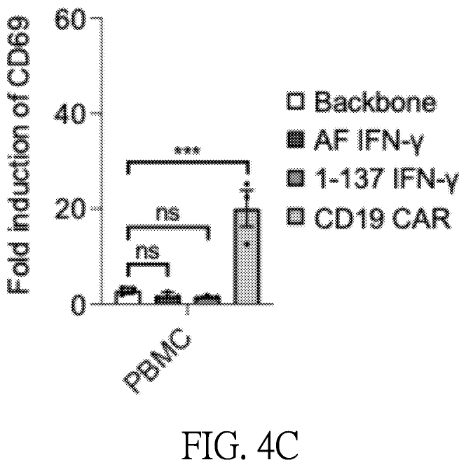

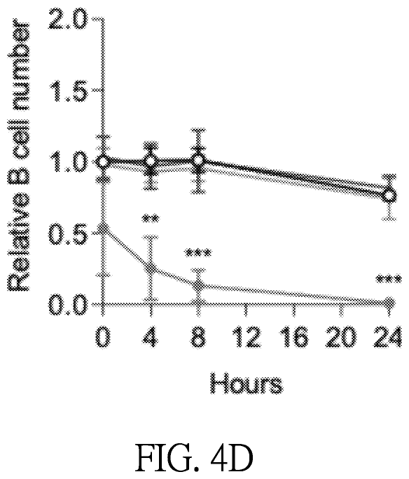

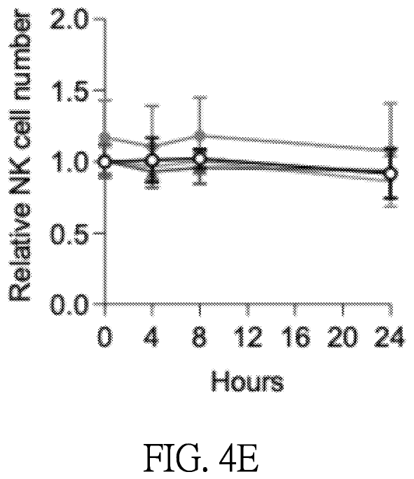

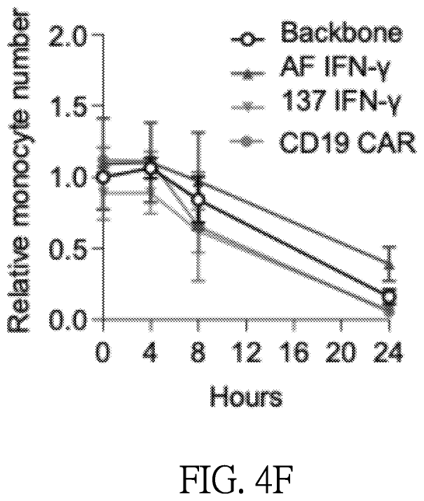

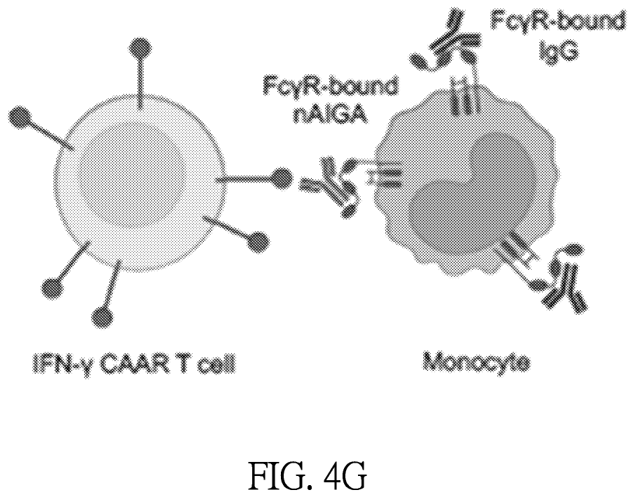

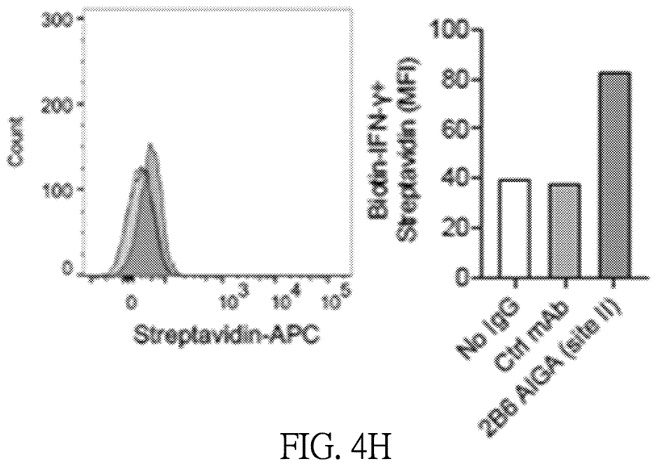

Despite the present application data indicating that truncated mutant forms of IFN-γ do not induce IFN-γR signaling, it remains possible that the weak binding between the present application IFN-γ variants and IFN-γR could lead to killing of IFN-γR-expressing cells. Additionally, IFN-γ is known to dimerize, raising the concern that IFN-γ CAARs could interact with endogenous IFN-γ secreted by activated T or NK cells, thereby facilitating IFN-γ CAAR cross-reactivity with IFN-γR. To evaluate these scenarios, as shown in FIG. 4A and FIG. 4B, the present application conducted co-culture experiments using K562 cells overexpressing either IFN-γR1 or the 2B6 nAIGA BCR and CD19, alongside wild-type K562 cells, with control T cells or IFN-γ CAAR T cells. Both AF and 1-137 IFN-γ CAAR T cells displayed no changes in CD69 expression or cytotoxicity against K562 cell line overexpressing IFN-γR1. Furthermore, the present application excluded that dimerization occurs between soluble IFN-γ with AF or 1-137 IFN-γ CAAR expressed on the primary T cells.

As shown in FIG. 4C, to examine the response of IFN-γ CAAR T cells to primary cells expressing IFN-γR, the present application employed autologous PBMCs as target cells. AF and 1-137 IFN-γ CAAR T cells did not increase CD69 after engaging PBMCs, but remained capable of inducing CD69 expression upon encountering K562 cells expressing the 2B6 nAIGA BCR. Moreover, as shown in FIG. 4D, CD19 CAR T cells killed primary B cells right after their encounter (0-hour time point) and eliminated all B cells by 24 hours later.

In contrast, B cells remained intact during 24 hours of co-culture with AF and 1-137 IFN-γ CAAR T cells. NK cell and monocyte numbers were also unaffected when incubated with both IFN-γ CAAR T cells (FIG. 4E-F). Together, these results indicate that the IFN-γ CAAR T cells the present application developed here do not eliminate cells with physiological expression levels of IFN-γR.

As shown in FIGS. 4G to 4I, Cells expressing the FcγIa receptor, such as monocytes and macrophages, can capture immunoglobulin G (IgG) in circulation with a high affinity. Therefore, the decoration of nAIGA on cells highly expressing FcγIa receptor in patients may have the potential to trigger Fc-redirected cytotoxicity upon encountering IFN-γ CAAR T cells. The present application next evaluated this possibility by using autologous primary monocytes as target cells after labeling them with serum IgG from healthy donors or patients with nAIGAs. The present application first confirmed that 2B6 nAIGA-labeled monocytes can capture biotinylated IFN-γ, but not control mAb-labeled monocytes, suggesting that monocytes indeed are decorated with nAIGA to capture IFN-γ. Next, the present application incubated IFN-γ CAAR T cells with monocytes decorated by serum from either healthy donors or patients with nAIGAs. The present application did not observe cytotoxic events among monocytes labeled with either the control or AIGA serum by IFN-γ CAAR T cells. However, monocytes labeled with anti-CD3 antibody (clone OKT3) served as a positive control, which were eliminated by CAAR backbone-expressing human T cells and IFN-γ CAAR T cells. Taken together, the present application data indicate that the physiological composition of nAIGAs in serum was unlikely to induce Fc-redirected killing.

Embodiment 5: IFN-γ CAAR T cells eliminate B cells derived from patients with nAIGA.

To further validate the efficacy of AF and 1-137 IFN-γ CAAR T cells on eliminating autoreactive B cells in nAIGA patients, the present application recruited four nAIGA patients experiencing nontuberculous mycobacterial infection (NTM P). The present application generated the IFN-γ CAAR T cells from patient-derived T cells. The IFN-γ CAAR expression pattern between nAIGA patient- and healthy donor-generated IFN-γ CAAR T cells was comparable.

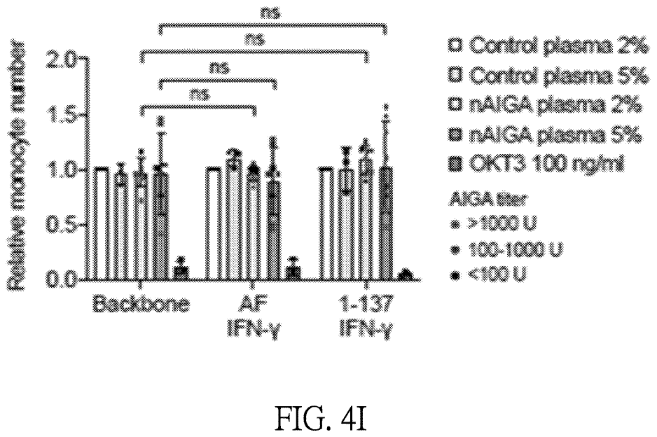

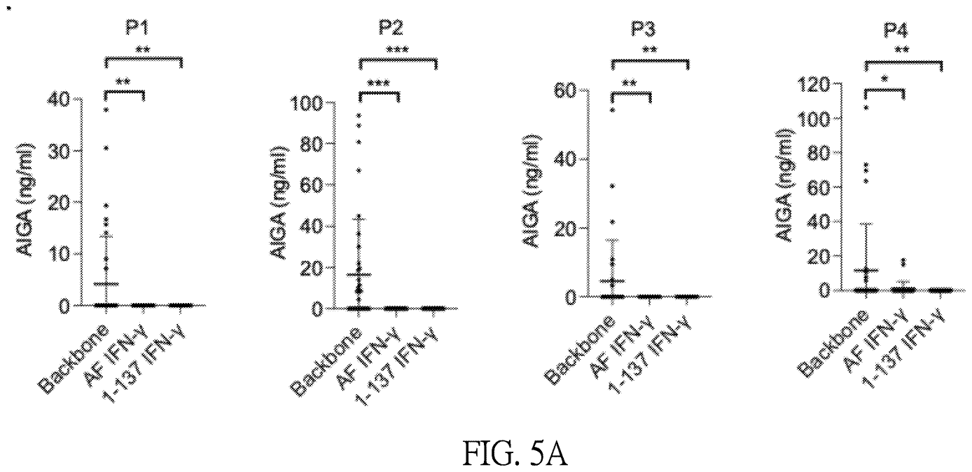

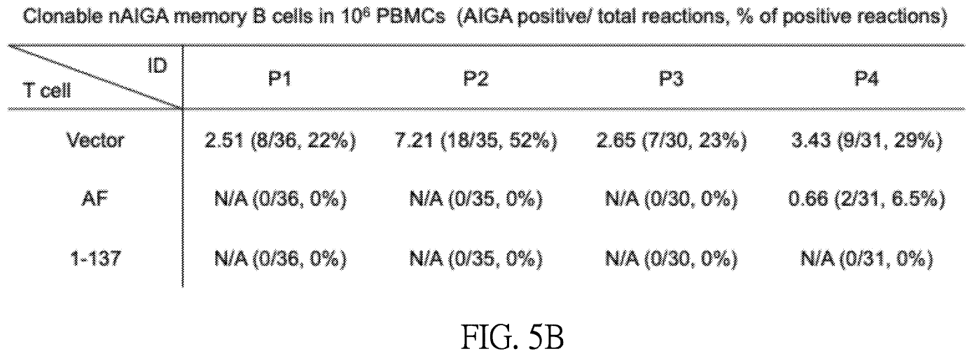

As shown in FIG. 5A, the present application co-cultured IFN-γ CAAR T cells with autologous PBMCs containing autoreactive B cells against IFN-γ (nAIGA B cells) ex vivo. IFN-γ CAAR T cell treatment robustly decreased the number of nAIGA antibody-positive wells compared to control T cell treatment in all four patient samples. As shown in FIG. 5B, Since the number of nAIGA B cells is very few in circulation (1 in 10⁶ PBMCs according to previous B cell cloning data (25)), the present application assessed the efficacy of IFN-γ CAAR T cell treatment on eliminating nAIGA B cell clones that can be differentiated from PBMCs. The present application successfully differentiated nAIGA B cell clones from these four patients' PBMCs and found 2.51, 7.21, 2.65, and 3.43 nAIGA B cell clones per 10⁶ circulating PBMCs in NTM P1-P4, respectively.

Surprisingly, AF IFN-γ CAAR T cells depleted most of the nAIGA B cell clones in all patients, except for NTM P4, in which 0.66 clones per 10⁶ circulating PBMCs were generated. In contrast, 1-137 IFN-γ CAAR T cells could eliminate all AIGA B cell clones among four patients. These results indicate that the nAIGA B cells in the circulation of NTM P4 may contain clones targeting site I epitope and highlight the potential of 1-137 IFN-γ CAAR T cells on covering the full spectrum of nAIGA clone diversity.

Embodiment 6: Develop receptor-unresponsive GM-CSF CAARs to target autoreactive B cells of GM-CSF.

Aim 1: To design a GM-CSF CAAR that can recognize anti-GM-CSF B cell receptor (BCR) but is unresponsive to endogenous GM-CSF receptor.

Aim 1.1 Screening for GM-CSF receptor-irresponsive GM-CSF variant as CAAR.

Unique anti-GM-CSF BCR expressed on the autoreactive B cells from patients with anti-GM-CSF autoantibody can be used as a targeted antigen. The present application will utilize GM-CSF as a chimeric autoantibody receptor to target anti-GM-CSF BCR expressed on the autoreactive B cells from patients with anti-GM-CSF autoantibodies. Specifically, the present application involves introducing GM-CSF CAAR variants into NFAT luciferase reporter Jurkat cells by lentivirus transduction. These Jurkat cells will be stimulated with coated GMRa or GMR-enriched cell lines such as TF-1 or Nalm-6 and their activation will be assessed by the induction of luciferase activity. The present application anticipates that the mutations will abolish the GMR cross reactivity by candidate GM-CSF CAARs.

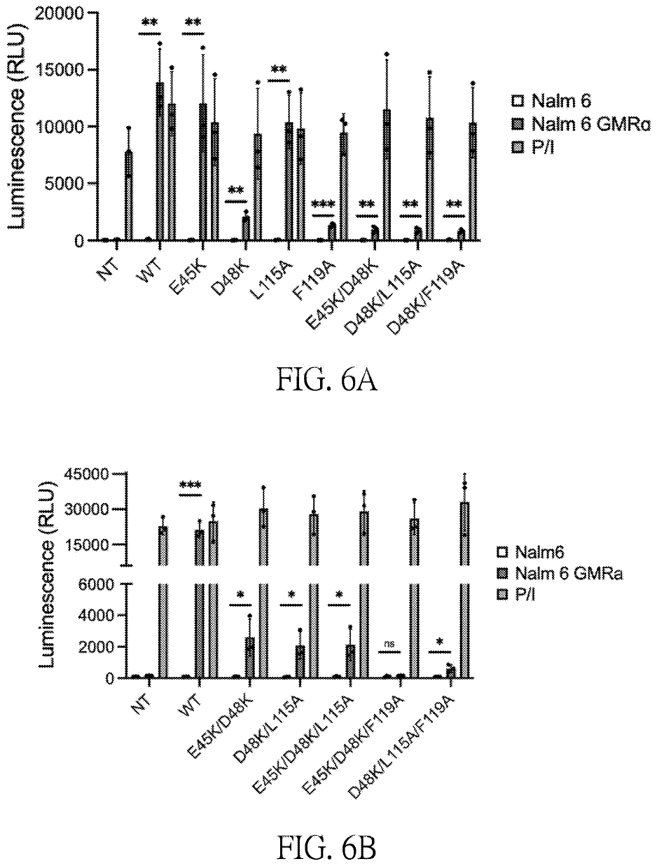

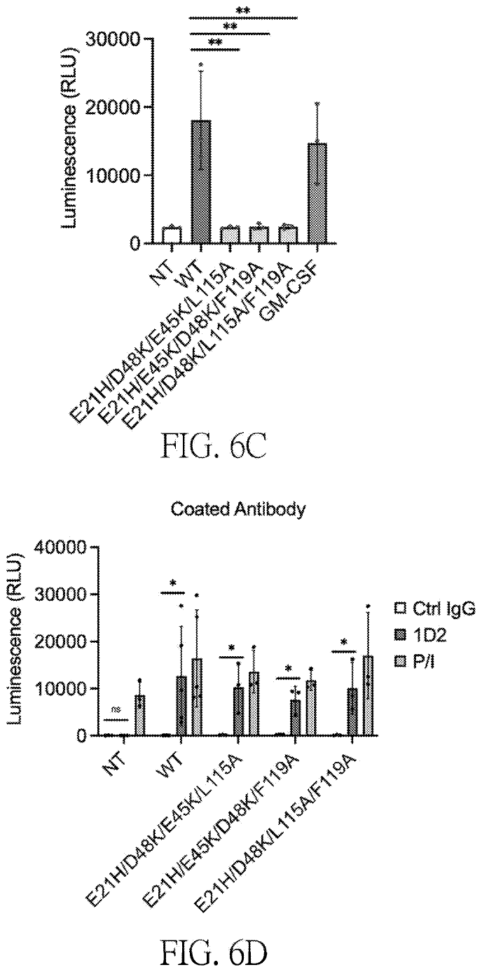

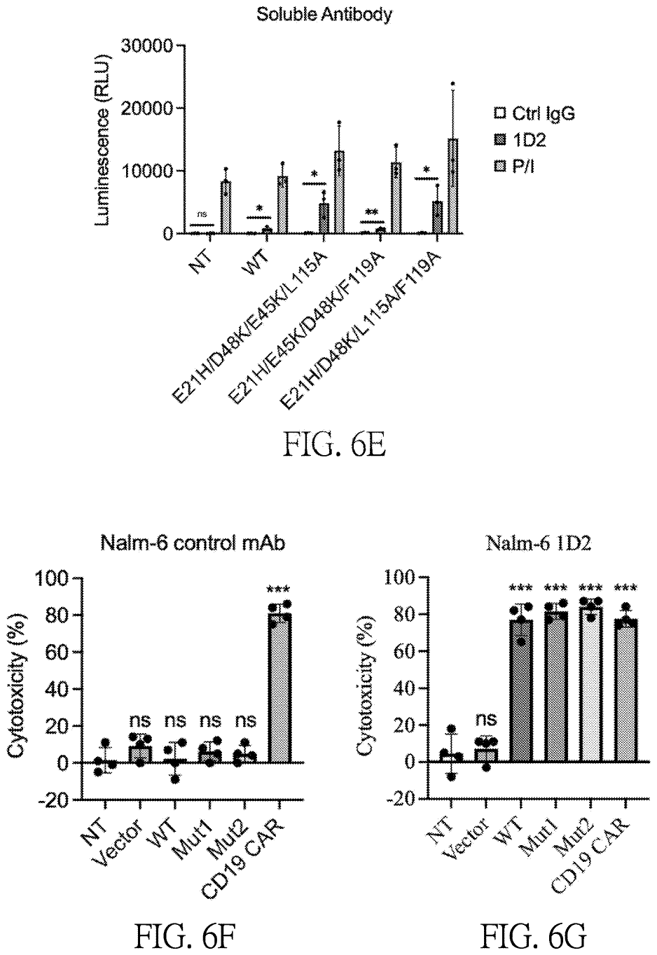

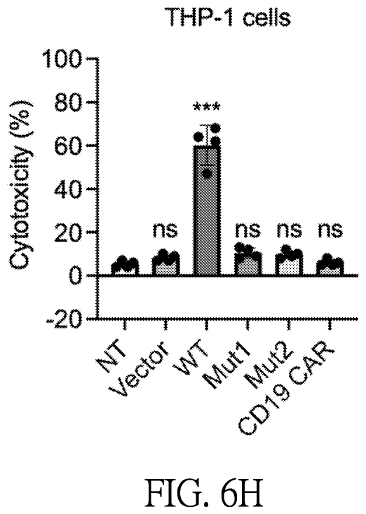

As shown in FIGS. 6A and 6B, Activation of Jurkat NFAT reporter cells expressing GM-CSF CAAR variants stimulated by GMRa-overexpressing Nalm-6 cells. A two-step screen was performed to identify signaling-inert GM-CSF CAAR variants carrying (FIG. 6A) 1 to 2 or (FIG. 6B) 2 to 3 mutations. PMA and ionomycin (P/I) treatment served as the positive control. Data are presented as mean±s.d. (n=3). Statistical significance was determined by an unpaired two-tailed t-test.

Aim 1.2 Screening for GM-CSF variant recognizing anti-GM-CSF autoantibody BCR as CAAR.

The exact epitopes for each group of anti-GM-CSF autoantibody are not clearly defined, and it's critical that the introduced point mutation in GM-CSF will not affect the interaction with anti-GM-CSF autoantibody. The NFAT luciferase reporter Jurkat cells expressing the candidate GM-CSF CAAR will be stimulated with each group of monoclonal anti-GM-CSF autoantibodies. The coated anti-GM-CSF autoantibody on the plate will be used to mimic membrane-bound BCR. Moreover, the soluble anti-GM-CSF autoantibody will be used to examine whether the GMCSF CAAR can discriminate the anti-GM-CSF BCR from soluble anti-GM-CSF autoantibody or not. The activation of GM-CSF CAAR will be evaluated by the induction of luciferase activity. The present application is expected to identify the GM-CSF CAAR that can only be activated by coated anti-GM-CSF autoantibody from the GM-CSF CAAR candidates that don't respond to GMR.

As shown in FIG. 6C, activation of U937 STAT5RE reporter cells stimulated by Jurkat cells expressing GM-CSF CAAR variants. GM-CSF treatment served as the positive control. Data are presented as mean±s.d. (n=3). Statistical significance was determined by One-Way ANOVA with Tukey's multiple comparisons test.

As shown in FIGS. 6D and 6E, activation of Jurkat NFAT reporter cells expressing signaling-inert GM-CSF CAAR variants stimulated by (FIG. 6D) plate-coated or (FIG. 6E) soluble anti-GM-CSF autoantibody. The patient-derived monoclonal antibody 1D2 was used as the specific autoantibody. PMA and ionomycin (P/I) treatment served as the positive control. Data are presented as mean±s.d. (n=3-5). Statistical significance was determined by an unpaired two-tailed t-test.

We aim to generate GM-CSF CAAR T cells that can discriminate anti-GM-CSF autoantibody BCR from endogenous GM-CSF receptors. In addition, the GM-CSF CAAR candidates should exhibit minimal activation by soluble anti-GM-CSF autoantibody.

Aim 2. To characterize the potency and persistence of GM-CSF CAAR T cells.

Aim 2.1 Screening for the optimal extracellular hinge for GM-CSF CAAR.

The extracellular hinge of CAR affects its sensitivity, activation capacity, and tonic signaling. Therefore, the selection of the most compatible hinge for GM-CSF CAAR is critical for its application. The activity of GM-CSF CAAR with extracellular hinges, including long IgG2, short IgG2, CD8, and CD28, will be investigated.

GM-CSF CAAR with long IgG2, short IgG2, CD8, and CD28 extracellular hinge will be introduced into human primary T cells by lentivirus, and their response to soluble anti-GM-CSF autoantibody, activation potency, and tonic signaling will be assessed. Upregulation of early activation marker CD69 will be adopted to evaluate the activation status of non-treated, soluble anti-GM-CSF autoantibody-treated, and coated anti-GM-CSF autoantibody-treated GM-CSF CAAR T cells. The present application is expected to identify GM-CSF CAARs that exhibit minimal tonic signaling and response to soluble anti-GM-CSF autoantibody but robust activation upon coated anti-GM-CSF autoantibody stimulation.

Aim 2.2 Characterization of the cytotoxicity, cytokine secretion ability, and proliferation capacity of GM-CSF CAAR T cells when engaging with anti-GM-CSF autoantibody BCR target cells in vitro.

Our goal is to study the fundamental T cell reactions when exposed to the anti-GMCSF BCR target cells. These reactions encompass cytotoxicity, proliferation capacity, and cytokine secretion ability. Any shortcomings observed in the T cell responses will indicate areas for improvement in the GM-CSF CAAR design, enabling us to enhance the engineering of the GM-CSF CAAR construct.

For evaluation of cytotoxicity of GM-CSF CAAR T cells, a flow cytometry-based cytotoxicity assay will be performed with a series of E:T ratios from 1:1, 3:1, 10:1, to 30:1. For evaluation of proliferation capacity, a cell proliferation assay based on dye dilution will be performed. Ctrl, GM-CSF CAAR, and FMC63-BBZ will be labeled with Celltrace reagent and react with the above-mentioned target cells for 72 hours, and the percentage of T cells that underwent proliferation will be analyzed by flow cytometry. For evaluation of the cytokine secretion profile, the conditioned medium from the proliferation assay will be collected after 24 hours of reaction and the representative cytokines such as interleukin-2, IFN-γ, and tumor necrosis factor-α (TNF-α) will be analyzed by enzyme-linked immunosorbent assay (ELISA). The optimal IFN-γ CAAR will be adopted for safety and in vivo validation.

As shown in FIGS. 6F to 6H, cytotoxicity of GM-CSF CAAR T cells against Nalm-6 cells expressing a control mAb or a patient-derived anti-GM-CSF autoantibody BCR (clone 1D2) (n=4 donors). Data are presented as mean±s.d. Statistical significance was determined by One-Way ANOVA. Wherein, 1D2 represents the anti-GM-CSF monoclonal antibody isolated from the patient, Mut1 represents E21H/E45L/D48K/L115A, and Mut2 represents E21H/D48K/L115A/F119A.

Thus, the present invention provides specific target B cells that generate anti-GM-CSF autoantibodies, and GM-CSF CAAR T cells do not exhibit GMR cross-reactivity toxicity.

Aim 2.3 Validation of efficacy to deplete anti-GM-CSF autoantibody BCR target cells of GM-CSF CAAR T cells in vivo.

The tumor microenvironment can influence the cytotoxicity of CAR T cells. Likewise, when GM-CSF CAAR T cells encounter anti-GM-CSF memory B cells in different tissues like the bone marrow or lymph nodes, similar challenges may arise. Therefore, the present application goal is to conduct in vivo experiments to confirm the cytotoxicity of GM-CSF CAAR T cells.

GM-CSF CAAR T cells will be transferred into NSG mice engrafted with anti-GM-CSF BCR Nalm-6 cells, and the control of tumor burden will be monitored by IVIS and confirmed by the reduction of Nalm-6 cell number in the bone marrow and spleen. Infiltration of GM-CSF CAAR T cells into the bone marrow and spleen, where the anti-GMCSF BCR Nalm-6 target cells reside, will be examined. In addition, a series of GM-CSF CAAR T cells ranging from 2*106, 5*106, 107, to 2 times 10⁷ cells will be tested for determining the minimal infused T cell number to eradicate anti-GM-CSF BCR Nalm-6 cells. The present application anticipates that the GM-CSF CAAR T cells will control the anti-GM-CSF BCR Nalm-6 progression and infiltrate into the bone marrow and spleen.

Aim 2.4 Evaluation of the persistence of GM-CSF CAAR T cells in vivo.

CAR T cells within their bodies. Furthermore, CAR T cells that possess a less differentiated status have been observed to exhibit prolonged persistence in these patients. The present application objective is to closely monitor the in vivo dynamics of IFN-γ CAR T cell numbers and their memory status.

GM-CSF CAAR T cells will be transferred into an anti-GM-CSF BCR Nalm-6 xenograft NSG mouse model, and the blood will be collected to analyze the number of GMCSF CAAR T cells by flow-cytometry. The memory status, including stem cell memory (TSCM, CD95⁺CD45RA⁺CCR7⁺), central memory (TCM, CD45RA⁻CCR7⁺), effector memory (TEM, CD45RA⁻CCR7⁻), and terminally differentiated effector memory cells re-expressing CD45RA (TEMRA, CD45RA⁺CCR7⁻), will be examined. The present application anticipates that the number of T cells will peak 1 week after infusion and gradually decrease. In addition, the persistence of GM-CSF CAAR T cells correlates with the percentage of T_{SCM} and T_{CM}.

Aim 2.5 Investigation of interference from soluble anti-GM-CSF autoantibody on the cytotoxicity of GM-CSF CAAR T cells against anti-GM-CSF autoantibody BCR target cells in vitro.

Soluble anti-GM-CSF autoantibody in circulation can bind to GM-CSF CAARs and may interfere with their interaction with anti-GM-CSF BCR expressed on the target cells. The titer of soluble anti-GM-CSF autoantibodies in the patient's plasma ranges from 8.8 to 189.4 units in the present application cohort study (2, 26) and is very likely to dampen the cytotoxicity of GM-CSF CAAR T cells. However, the avidity of the anti-GM-CSF autoantibody mixture in patients is not determined, and their interference should be confirmed by experiment.

A flow cytometry-based cytotoxicity assay will be conducted to assess the efficacy of GM-CSF CAAR T cells to eliminate anti-GM-CSF BCR target in the presence of soluble anti-GM-CSF autoantibodies. The mixture of high-affinity monoclonal soluble anti-GM-CSF autoantibodies or the patient's plasma will be used to mimic the condition that GM-CSF CAAR T cells will encounter in the patient.

Aim 2.6 Establishment of mice carrying soluble anti-GM-CSF autoantibody in circulation to evaluate the interference of anti-GM-CSF autoantibody on the cytotoxicity of GM-CSF CAAR T cells against anti-GM-CSF autoantibody BCR target cells in vivo.

The presence of soluble anti-GM-CSF autoantibodies in circulation not only has the potential to dampen the cytotoxicity of GM-CSF CAAR T cells but also to interfere with their infiltration into tissues where B cells, such as bone marrow, spleen, and lymph nodes, are located. Additionally, GM-CSF CAAR T cells coated with soluble anti-GM-CSF autoantibodies may become susceptible to targeting by FcγR-enriched cells, such as myeloid cells.

Given these potential implications, it is essential to conduct an investigation to understand how soluble anti-GM-CSF autoantibodies affect the behavior and fate of GM-CSF CAAR T cells. Such research will provide valuable insights into the interaction between the autoantibodies and the therapeutic GM-CSF CAAR T cells, helping to optimize their effectiveness in clinical applications.

Anti-GM-CSF CAAR BCR Nalm-6 transduced with secretory anti-GMCSF autoantibody by lentivirus will be adopted in the Nalm-6 xenograft NSG mice model. The concentration of soluble anti-GM-CSF autoantibody in the circulation will be monitored weekly using human IgG ELISA. GM-CSF CAAR T cells will be transferred into the mice when detectable levels of soluble anti-GM-CSF autoantibody are observed. The progression of Nalm-6 cells and the persistence of T cells will be examined using the same experimental procedures as outlined in Aim 2.4. Additionally, the present application will track the dynamics of GM-CSF concentration in the circulation. The present application anticipates that soluble anti-GM-CSF autoantibodies will delay, but not compromise, the regression of Nalm-6 cells, and the circulatory anti-GM-CSF autoantibodies will diminish following GM-CSF CAAR T cell treatment.

Established Nalm-6 cells are hard to eliminate in vivo, and if the Nalm-6 cells can't secrete enough anti-GM-CSF autoantibody in a short time, the excessive Nalm-6 cells may confound the effect of soluble anti-GM-CSF autoantibodies on GM-CSF CAAR T cells. Alternatively, the present application will use self-generated monoclonal anti-GM-CSF autoantibodies and transfer them together with Nalm-6 cells to establish a Nalm-6 xenograft model carrying soluble anti-GM-CSF autoantibodies.

Regarding potential FcγR-mediated adverse effects on anti-GM-CSF autoantibody-coated GM-CSF CAAR T cells, the present application acknowledges that NSG mice are immunodeficient and lack functional macrophages and dendritic cells. To accurately reflect FcγR-mediated events, immunocompetent mice or humanized NSG mice can be utilized in the study. These alternative models would better represent the in vivo scenario and provide relevant insights into the interactions between GM-CSF CAAR T cells and the host immune system.

We aim to develop GM-CSF CAAR T cells that can specifically deplete anti-GM-CSF autoantibody BCR target cells and persist normally in vivo, even in the presence of circulating anti-GM-CSF autoantibodies.

Aim 3. To evaluate the safety and specificity of GM-CSF CAAR T cell.

Aim 3.1 To evaluate the GM-CSF cross-reactive toxicity by GM-CSF CAAR T cells in vitro.

GMR is highly expressed in several immune cells, especially in the myeloid lineage, and hence the cross-reactivity between GMR and GM-CSF CAAR should be carefully examined even though the GMR non-responsive IFN-γ variant is adopted.

Our goal is to assess the GMR cross-reactive toxicity of GM-CSF CAAR T cells using a flow cytometry-based cytotoxicity assay. Concurrently, the present application will monitor the activation of GM-CSF CAAR T cells by analyzing the upregulation of CD69. To study GMR-associated toxicity, the present application will create GMRa knockouts (KO) in GMR-expressing cell lines like Nalm-6 cells using CRISPR as target cells. Additionally, the present application will generate GMRa-overexpressed (OE) TF-1 and Nalm-6 cells as target cells.

We hypothesize that GM-CSF CAAR T cells will not be activated by GMRa OE TF-1 and Nalm-6 cells, and they will not exhibit any additional cytotoxicity towards these target cells. However, in case the present application observe GMR cross-reactive toxicity, the present application will introduce more critical mutations to further disrupt the interaction between GM-CSF CAAR and GMR. This approach will enable us to thoroughly investigate and understand the potential risks associated with GMR cross-reactivity and develop safer and more effective GM-CSF CAAR T cell therapies.

Aim 3.2 To validate the GM-CSF cross-reactive toxicity by GM-CSF CAAR T cells in vivo.

Inflammatory cytokines in tissue can lower the threshold of T cell receptor activation, which may also be applied for CAR activation. Therefore, GMR cross-reactivity by GM-CSF CAAR should be examined in vivo.

We aim to validate the absence of GMR cross-reactive toxicity by GMCSF CAAR T cells in vivo. The dual Nalm-6 xenograft model in NSG mice will be adopted to investigate the selective reactivity of GM-CSF CAAR T cells to anti-GM-CSF BCR Nalm-6 (on-target) from GMRa-OE CD19KO (off-target) Nalm-6. FMC63-BBZ will be included as a positive control for proper result interpretation. The present application anticipates that the GMRa-OE CD19KO (off-target) Nalm-6 will not be affected by GM-CSF CAAR T cells, while the anti-GM-CSF BCR Nalm-6 will be destroyed (on-target).

Aim 3.3 To analyze the specificity of GM-CSF CAAR T cells in the presence of soluble anti-GM-CSF autoantibody in vitro.

Soluble anti-GM-CSF autoantibody in circulation may directly activate IFN-γ CAAR T cells and induce non-specific toxicity to the surrounding cells.

Our objective is to investigate the non-specific toxicity induced by anti-GM-CSF autoantibodies using a flow-cytometry-based selective killing assay. In this study, the present application will co-culture control and anti-GM-CSF BCR Nalm-6 cells with GM-CSF CAAR T cells in the presence of soluble anti-GM-CSF autoantibody. For this purpose, the present application will use either a self-generated monoclonal anti-GM-CSF autoantibody or the patient's plasma as a source of anti-GM-CSF autoantibody.

We expect that the presence of soluble anti-GM-CSF autoantibodies will diminish the cytotoxicity of GM-CSF CAAR T cells against anti-GM-CSF BCR CD19 Nalm-6 cells, while the control CD19 KO Nalm-6 cells will remain unaffected. However, in the event of nonspecific toxicity induced by soluble anti-GM-CSF autoantibodies, the present application will explore the development of a SynNotch version of GM-CSF CAAR T cells. This modified version will limit the expression of GM-CSF CAAR until the T cells encounter B cells, thereby potentially mitigating any non-specific effects.

Aim 3.4 To examine the FcγR-redirected toxicity by GM-CSF CAAR T cells in vitro.

Cells highly expressing FcγR such as macrophages and monocytes, can capture IgG in circulation, and it's very likely that these cells from the patients are decorated with anti-GMCSF autoantibodies. The FcγR-redirected toxicity may be observed in anti-GM-CSF autoantibody-coated cells.

Our aim is to investigate the possibility of FcγR-redirected killing by GMCSF CAAR T cells using a flow-cytometry cytotoxicity assay. To achieve this, the present application will isolate monocytes and coat them with anti-GM-CSF autoantibodies obtained from self-generated monoclonal anti-GM-CSF autoantibodies or the patient's plasma. As a positive control, the present application will use the mouse anti-human CD3 antibody (clone: OKT3).

To evaluate the labeling efficiency, the present application will stain the coated monocytes with biotinylated GMCSF and either fluorophore-conjugated streptavidin or fluorophore-conjugated anti-mouse IgG (H+L) antibody. The flow-cytometry analysis will be performed to assess successful IgG capture by FcγR.

We expect that FcγR-redirected toxicity by GM-CSF CAAR T cells will be absent, as observed in Dsg3 CAAR T cells (4). Nevertheless, if FcγR-redirected toxicity is severe, a SynNotch version of GM-CSF CAAR T cells can also solve the problem.

Aim 3.5 To investigate the general toxicity to peripheral blood mononuclear cells by GMCSF CAAR T cells in vitro.

Considering that primary cells may respond differently to GM-CSF CAAR T cell-associated toxicity compared to cancer cell lines, it is important to assess the potential adverse effects on primary cells. Peripheral blood mononuclear cells (PBMCs) are accessible and express GMR. The above-mentioned toxicity should be examined by using PBMCs as target cells.

We aim to investigate general toxicity from GM-CSF CAAR T cells to PBMCs by flow-cytometry cytotoxicity assay. The FMC63-BBZ CAR T cells will be included in the experiment as a positive control. Autologous PBMCs will be isolated and co-cultured with GM-CSF CAAR T cells, the present application will monitor the general toxicity at 0, 4, 8, and 24-hour time points using flow cytometry. Additionally, the present application will examine the activation of GM-CSF CAAR T cells at the 24-hour time point by analyzing CD69 up-regulation with flow cytometry.

We anticipate that GM-CSF CAAR T cells will not exert significant toxicity towards PBMCs.

Aim 3.6 To assess the general toxicity of GM-CSF CAAR T cells in vivo.

The diverse microenvironment in each tissue makes it challenging to predict the interaction of GM-CSF CAAR T cells. Additionally, the sensitivity of cells in different tissues to potential toxicity from GM-CSF CAAR T cells may vary, necessitating careful examination.

To examine the general toxicity from GM-CSF CAAR T cells in vivo, major organs, including the heart, liver, spleen, lung, kidney, and colon, will be collected 7 days (acute) and 15 days (subacute) post-GM-CSF CAAR T cells infusion from NSG mice. These organs will be processed for histology analysis. The present application anticipates observing the infiltration of GMCSF CAAR T cells in the lung, liver, and spleen during the acute phase, but primarily in the spleen during the subacute phase. Moreover, the present application do not expect to observe severe tissue damage in any of the collected organs.

Our objective is to conduct a comprehensive assessment of the safety profile of GM-CSF CAAR T cells and identify any potential undesired toxicity. This thorough characterization will enable us to make necessary modifications and improvements, ensuring the safety of GM-CSF CAAR T cell therapy.

Aim 4. To validate the feasibility of depleting patient-derived autoreactive B cells against GM-CSF by GM-CSF CAAR T cells.

Aim 4.1 To develop an epitope-specific anti-GM-CSF autoantibody ELISA.

Anti-GM-CSF autoantibody can lead to two exclusive manifestations: autoimmune PAP and anti-GM cryptococcal meningitis. In the present application cohort, the present application didn't observe any difference in the anti-GM-CSF autoantibody titer, neutralizing ability, and immunoglobulin isotype between the two diseases. Considering the binding epitope can determine the neutralizing ability and Fc-dependent function of antibodies, the present application speculates that the composition of anti-GM-CSF autoantibodies may be different between the two diseases, and dissecting this difference could provide additional insight into anti-GM-CSF autoantibody-associated diseases.

We aim to analyze the composition of anti-GM-CSF autoantibodies in the patients and their relationship with the clinical manifestation. The present application will generate the human murine-swapped GM-CSF, which retains only one binding epitope of anti-GM-CSF autoantibody on murine GM-CSF. Specifically, 4 α-helix and two β-strands in GM-CSF will be manipulated. The selective reactivity of these GM-CSF variants will be confirmed by anti-GM-CSF autoantibody ELISA using monoclonal anti-GM-CSF autoantibody from each group. The present application anticipates that the epitope-specific anti-GM-CSF autoantibody ELISA can distinguish anti-GM-CSF autoantibodies from each group.

Aim 4.2 To determine the binding epitope of each anti-GM-CSF autoantibody clone ex vivo.

The ex vivo differentiation approach allows us to identify IgG+ B cell clones specific to GMCSF. This method not only helps us determine the frequency of these clones in circulation but also provides an excellent opportunity to study individual anti-GM-CSF autoantibodies in detail.

We will collect PBMCs from patients diagnosed with anti-GM-CSF autoantibody-associated PAP and cryptococcal meningitis. Subsequently, the present application will generate IgG+ B cell clones targeting GM-CSF using memory B cell differentiation conditions. Initially, the present application will identify the anti-GM-CSF autoantibody clones using a normal anti-GM-CSF autoantibody ELISA. Subsequently, the present application will employ an epitope-specific anti-GM-CSF autoantibody ELISA to determine the specific binding epitopes on GM-CSF for these clones. The present application analysis will focus on examining the correlation between the number of clonable IgG+ B cell clones against GM-CSF and the composition of anti-GM-CSF autoantibodies with the clinical manifestations displayed by the patients.

Possible difficulty and alternative approach: It is essential to acknowledge that autoreactive B cells targeting GM-CSF in circulation are rare and may vary between patients. Consequently, it might be challenging to identify enough anti-GM-CSF autoantibody clones for thorough analysis in some cases. As an alternative approach, the present application can still gain valuable insights by studying the anti-GM-CSF autoantibody composition in patients' plasma through epitope-specific anti-GM-CSF autoantibody ELISA. This alternative strategy will enable us to understand the immune response against GM-CSF and its association with the clinical manifestations observed in the patients.

Aim 4.3 To examine the efficacy of GM-CSF CAAR T cells to eliminate autoreactive B cells against GM-CSF from patients with anti-GM-CSF autoantibodies.

GM-CSF composition may be more diverse than the monoclonal anti-GM-CSF autoantibodies used in the present application to design the GM-CSF CAAR. Therefore, it is crucial to investigate the target coverage of GM-CSF CAAR T cells by patient-derived B cells against GM-CSF.

GM-CSF CAAR T cells will first be generated from the patient's T cells. Afterward, the patient's PBMCs will be co-cultured with autologous non-transduced or GMCSF T cells in the memory B cell differentiation medium. The effectiveness of depleting B cells targeting GM-CSF will be determined by comparing the reduction of anti-GM-CSF autoantibody clones generated from GM-CSF CAAR T cell treatment with that of non-transduced T cell treatment. Additionally, the total IgG production capacity in each reaction will be measured to assess any non-specific toxicity.

We anticipate observing a decrease in the number of anti-GM-CSF autoantibody clones under GM-CSF CAAR T cell treatment, while the overall IgG production ability remains unaffected.

Our study has two main goals. Firstly, the present application will analyze the composition of anti-GM-CSF autoantibodies in patients and investigate how they may influence the clinical manifestations observed in these individuals. Secondly, the present application will assess and confirm the efficacy of GM-CSF CAAR T cells in specifically targeting and eliminating autoreactive B cells derived from the patients.

Aim 5: To develop an in vivo delivery system of GM-CSF CAAR transgene to reprogram T cells in situ.

Aim 5.1 To evaluate the activity of lipid nanoparticle (NP)-transfected GM-CSF CAAR T-cell.

Our objective is to develop a T-cell targeting NP to engineer T cells in vivo. Delivery of GM-CSF CAAR mRNA in vivo can greatly decrease the manufacturing cost compared with traditional ex vivo expansion procedures. However, the lifespan of mRNA is short and hence multiple doses of NP are required to sustain the engineered T cell population in vivo. To design an optimal paradigm of NP injection, the dynamics of NP-transfected GM-CSF CAAR T cell population and their function should be characterized.

The GM-CSF CAAR expression in primary human T cells transfected by T-cell targeting NP with GM-CSF CAAR mRNA will be monitored by qPCR and flow cytometry until it is below the threshold of expression. The cytotoxicity and cytokine secretion ability will be characterized and compared with the lentivirus-transduced version. The present application expects the expression of the GM-CSF CAAR to peak 1-2 days post-transfection and then gradually diminish. In addition, the effector function of NP-transfected GM-CSF CAAR T cell is comparable with the lentivirus-transduced version in the short-term in vitro setting.

Aim 5.2 To analyze the efficacy of in vivo transcribed GM-CSF CAAR T cells.

Our goal is to deplete anti-GM-CSF BCR-targeted cells in vivo using in situ reprogrammed GM-CSF CAAR T cells. The transfection efficiency and stability of GM-CSF CAAR mRNA in T cells will affect the efficacy of GM-CSF CAAR T cells against the anti-GM-CSF BCR target cells. To optimize the NP injection protocol, the present application will evaluate the activity of in vivo transcribed GM-CSF CAAR T cells.

We will adopt the Nalm-6 xenograft NSG model to assess the effectiveness of in situ reprogrammed GM-CSF CAAR T cells. The lentivirus-transduced version will be included for side-by-side comparison. Human T cells are first intravenously injected into NSG, followed by NP injection at a certain period determined by Aim 5.1. Control of Nalm-6 progression is monitored by IVIS. The dynamics of in situ reprogrammed GM-CSF CAAR T cells are tracked by flow cytometry before and after every NP injection. The number of Nalm-6 and GM-CSF CAAR T cells in the bone marrow and spleen will be analyzed by flow cytometry at the end of the experiments. The present application expected the efficacy to control the Nalm-6 cell progression to be comparable between NP-transfected and lentivirus-transduced GM-CSF CAAR T cells.

Aim 5.3 To evaluate the specificity of T-cell-targeted lipid nanoparticles.

We will investigate cell types that are prone to off-target reprogramming by T cell-targeting NPs. Although the NPs are designed to specifically transfect the human T cells through scFV conjugated on the NP surface, endocytosis-active cells such as myeloid cell lineage can non-specifically uptake the NP.

The mouse T cell-targeting NPs and non-conjugated NPs equipped with tdtomato and EGFP mRNA, respectively, are injected intravenously into BALB/c mice, and the major organs such as the lung, heart, liver, spleen, kidney, thymus, lymph node, pancreas, and muscle are collected 48 hours after. The tdtomato signal in this organ will be detected by fluorescent IVIS. Meanwhile, immune cells transfected by NPs will be examined by flow cytometry.

Anticipated results include higher tdtomato signals in T cell-enriched organs like the spleen, thymus, and lymph nodes when compared to other organs, where EGFP signals will be less likely to be detected. Furthermore, T cells are expected to be the primary cell type displaying the tdtomato signal, with only a small fraction of macrophages possibly showing both tdtomato and EGFP signals.

Aim 5.4 To evaluate the toxicity of T-cell-targeted lipid nanoparticles.

NPs may induce unwanted immune reactions. Characterization of the safety profile is critical for the clinical application.

The 6-8-week-old Sprague-Dawley rat is utilized to assess the potential infusion reaction of NPs. The in vitro toxicity assays using nanoparticles were conducted following the standardized analytical cascade assays, as described on the NCL (Nanotechnology Characterization Laboratory). Moreover, histology examination of the major organs and hematology test will be conducted.

We aim to develop T-cell-targeted lipid nanoparticles for generating GMCSF CAAR T cells in situ. The efficacy of in vivo transcribed GM-CSF CAAR T cells in the depletion of anti-GM-CSF autoantibody BCR target cells will be analyzed. The specificity and toxicity of T-cell-targeted lipid nanoparticles will be carefully examined.

Embodiment 7: Develop receptor-unresponsive IFN-α CAARs to target IFN-α autoreactive B cells.

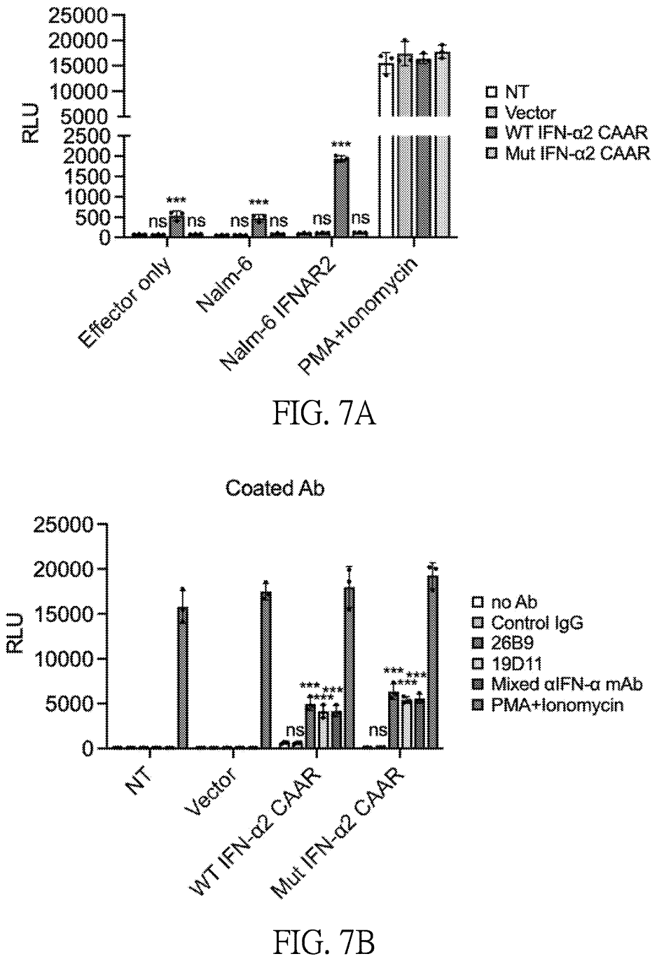

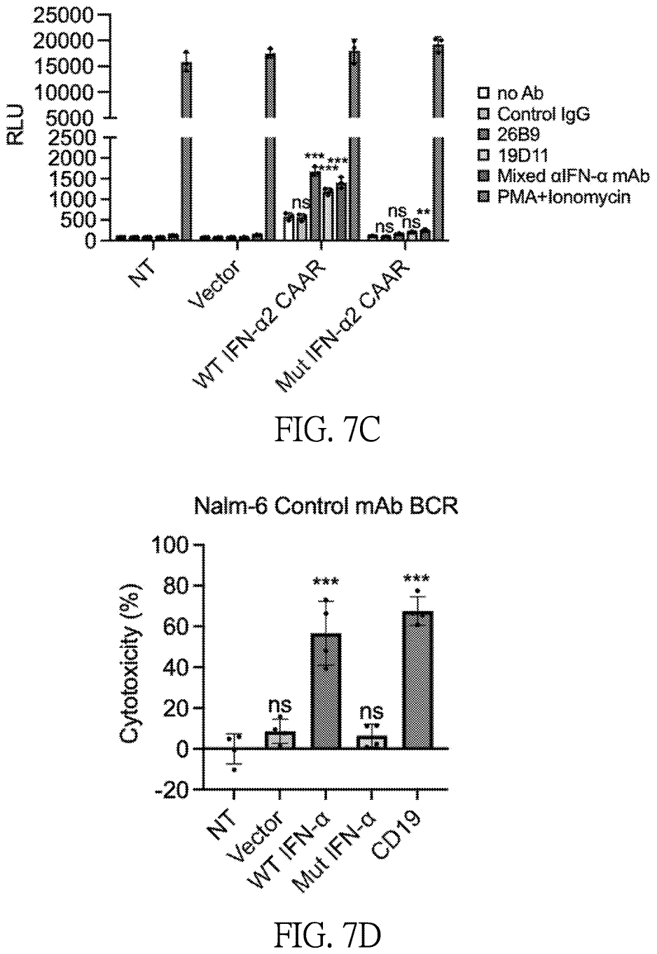

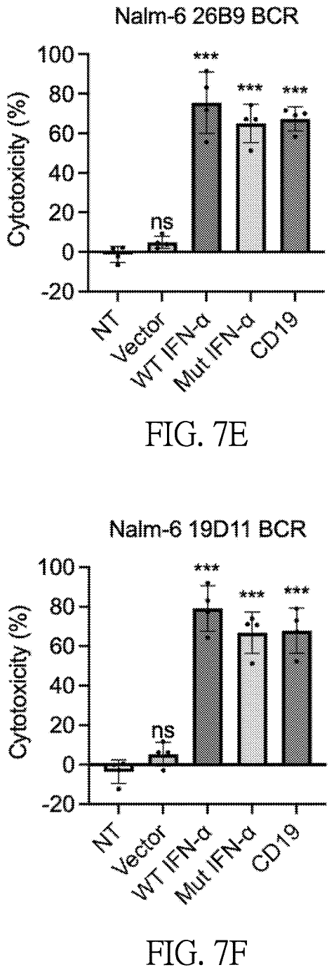

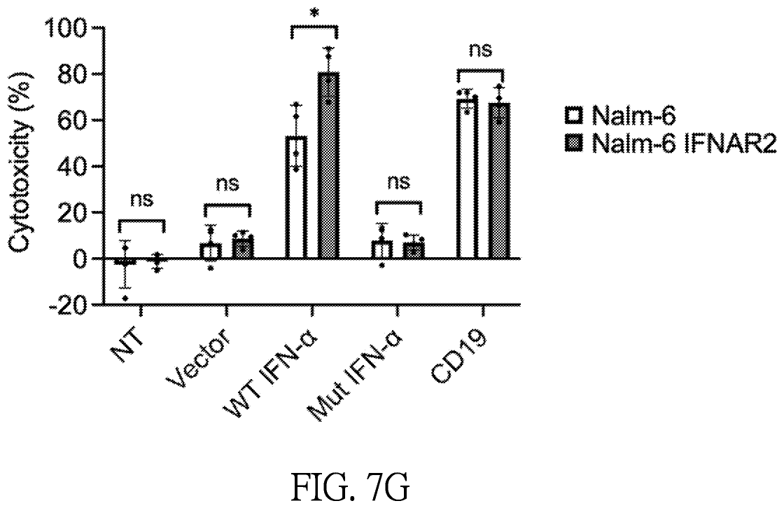

In a similar architecture to Embodiment 6, the present invention utilizes patient-derived monoclonal anti-type I interferon autoantibodies to evaluate mutant IFN-α degraders. As shown in FIG. 7A, activation of Jurkat NFAT reporter cells expressing IFN-α CAAR variants stimulated by IFNAR2-overexpressing Nalm-6 cells. PMA and ionomycin (P/I) treatment served as the positive control. Data are presented as mean±s.d. (n=3). Statistical significance between NT and other treatments within each group was determined using One-Way ANOVA with a Tukey post hoc test.

Referring to FIGS. 7B and 7C, activation of Jurkat NFAT reporter cells expressing signaling-inert IFN-α CAAR variants stimulated by (FIG. 7B) plate-coated or (FIG. 7C) soluble anti-IFN-α autoantibody. The patient-derived monoclonal antibody 26B9 and 19D11 was used as the specific autoantibodies. PMA and ionomycin (P/I) treatment served as the positive control. Data are presented as mean±s.d. (n=3). Statistical significance between NT and other treatments in each group was determined by One-Way ANOVA with Tukey post hoc analysis.

Referring to FIGS. 7D to 7F, cytotoxicity of IFN-α CAAR T cells against Nalm-6 cells expressing a (FIG. 7D) control mAb or a patient-derived anti-IFN-α autoantibody BCR clone (FIG. 7E) 26B9 and (FIG. 7F) 19D11 (n=4 donors). Data are presented as mean±s.d. Statistical significance between NT and other treatments was determined using One-Way ANOVA with a Tukey post hoc analysis.

Referring to FIG. 7G, in the Luciferase-based cytotoxicity assay, IFNAR2 receptor cross-reactive toxicity of IFN-α CAAR T cells against Nalm-6 cells overexpressing IFNAR2 (n=4 donors) . . . . Data are presented as mean±s.d. Statistical significance was determined by unpaired samples T-test (two-tailed).

Thus, this invention provides B cells that specifically target and produce autoantibodies against type I interferon. Furthermore, IFN-α CAAR T cells do not produce IFNAR cross-reactivity toxicity.

As an embodiment of the present invention, the sequence used is described in Table 1 below. For detailed sequences, please refer to the sequence table.

TABLE 1

| SEQ ID NO | describtion |
|---|---|
| 1 | CAARbackbone |
| 2 | WT IFN-γ CAAR - full length |
| 3 | A23Q IFN-γ CAAR - full length |
| 4 | H111D IFN-γ CAAR - full length |
| 5 | A23Q H111D IFN-γ CAAR - full length |
| 6 | A23Q H111D IFN-γ CAAR - Full length without |
| | signaling domain |
| 7 | A23Q H111D IFN-γ CAAR - AB |
| 8 | A23Q H111D IFN-γ CAAR - CE |
| 9 | A23Q H111D IFN-γ CAAR - Ct |
| 10 | A23Q H111D IFN-γ CAAR - ABLCt |
| 11 | A23QH111D IFN-γ CAAR - 1-131 |
| 12 | A23Q H111D IFN-γ CAAR - 1-134 |
| 13 | A23Q H111D IFN-γ CAAR - 1-135 |
| 14 | A23Q H111D IFN-γ CAAR - 1-136 |
| 15 | A23Q H111D IFN-γ CAAR - 1-137 |
| 16 | A23Q H111D IFN-γ CAAR - 1-138 |
| 17 | A23Q H111D IFN-γ CAAR - 1-142 |
| 18 | A23Q H111D IFN-γ CAAR -Full length dimer |
| | 1xG4S |
| 19 | WT GM-CSF CAAR: |
| 20 | E45K GM-CSF CAAR |
| 21 | D48K GM-CSF CAAR |
| 22 | L115A GM-CSF CAAR |
| 23 | F119A GM-CSF CAAR |
| 24 | E45K D48K GM-CSF CAAR |
| 25 | D48K L115A GM-CSF CAAR |
| 26 | D48K F119A GM-CSF CAAR |
| 27 | E45K D48K L115A GM-CSF CAAR |
| 28 | E45L D48K F119A GM-CSF CAAR |
| 29 | D48K L115A F119A GM-CSF CAAR |
| 30 | E21H GM-CSF CAAR |
| 31 | E21K GM-CSF CAAR |
| 32 | E21R GM-CSF CAAR |
| 33 | E21H E45K D48K L115A GM-CSF CAAR |
| 34 | E21H E45K D48K F119A GM-CSF CAAR |
| 35 | E21H D48K L115A F119A GM-CSFCAAR |
| 36 | WT IFN-α CAAR |
| 37 | R33A R120E IFN-α CAAR |

### Beneficial Effects of the Embodiments

In the present disclosure, by virtue of “a CAAR backbone as shown in SEQ ID no: 1” and “an antigen recognition domain includes a modified autoantigen or cytokine” to enhance the efficacy of chimeric antigen receptor T cells in the treatment of autoimmune diseases and their applications.

Furthermore, the present invention provides an autologous antibody monoclonal isolation platform for isolating multiple high-affinity anti-gamma interferon antibodies, which primarily block the assembly process of the gamma interferon receptor. This type of functional analysis further enhances its feasibility as a potential therapeutic tool. On the other hand, the antibodies isolated from patients in this way are antibodies derived from all of humanity, possessing high affinity and physiological activity, and are suitable for development into drugs to treat diseases caused by excessive gamma interferon.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A chimeric antigen receptor T cell, comprising:
i) a CAAR backbone as shown in SEQ ID no: 1; and
ii) an antigen recognition domain includes a modified autoantigen or cytokine;
wherein the autoantigen or cytokine includes one or more amino acid substitutions, deletions, and/or insertions that completely abolish the biological activity of the corresponding wild-type autoantigen or cytokine, while minimizing any reduction in binding to the autoantibody.

2. The chimeric antigen receptor T cell according to claim 1, wherein the cytokine is interferon-γ (IFN-γ), interferon-α (IFN-α) or granulocyte-macrophage colony-stimulating factor (GM-CSF).

3. The chimeric antigen receptor T cell according to claim 1, wherein the chimeric antigen receptor T cell comprises a chimeric autoantibody receptor (CAAR) with double mutations of A23Q and H111D.

4. The chimeric antigen receptor T cell according to claim 1, wherein the chimeric antigen receptor T cell maintained surface expression in IFN-γ CAAR containing 1-137 amino acids, and exhibited the strongest cytotoxicity in IFN-γ CAAR with a C-terminal extension of 1-131 to 1-142 amino acids.

5. The chimeric antigen receptor T cell according to claim 1, wherein the chimeric antigen receptor T cell eliminate patient-derived autoreactive B cell against IFN-γ.

6. The chimeric antigen receptor T cell according to claim 2, wherein, among variants of the granulocyte-macrophage colony-stimulating factor, a full-length monomeric GM-CSF with mutations in E21H, D45K, L115A, and F119A completely eliminated the interaction with GMR and the induction of GM-CSF signaling.

7. The chimeric antigen receptor T cell according to claim 2, wherein, among variants of the granulocyte-macrophage colony-stimulating factor, a full-length monomeric GM-CSF with E21H, D48K, L115A, and F119A mutations retains reactivity to patient-derived GM-CSF autoantibodies.

8. The chimeric antigen receptor T cell according to claim 2, wherein, in the interferon-α, a full-length monomeric IFN-α with R33A and R120E mutations completely eliminates the interaction with IFNAR.

9. The chimeric antigen receptor T cell according to claim 2, wherein, in the interferon-α, a full-length monomeric IFN-α with R33A and R120E mutations is still react with patient-derived anti-IFN-α autoantibodies.

10. Use of a chimeric antigen receptor T cell according to claim 1 for the preparation of a pharmaceutical composition for treating autoantibody-mediated diseases.
